(19) United States
(12) Patent Application Publication
(10) Pub. No.: US 2026/0140117 A1
(43) Pub. Date: May 21, 2026

(54) Methods for Diagnosing and Treating Vestibular Schwannoma

(71) Applicant: The Board of Trustees of the Leland Stanford Junior University, Stanford, CA (US)

(72) Inventors: Konstantina Stankovic, Redwood City, CA (US); Sasa Vasilijic, Redwood City, CA (US)

(21) Appl. No.: 19/120,958

(22) PCT Filed: Nov. 10, 2023
(86) PCT No.: PCT/US2023/079376

§ 371 Date: Apr. 14, 2025

Related U.S. Application Data
(60) Provisional application No. 63/383,407, filed on Nov. 11, 2022.
(60) Provisional application No. 63/518,820, filed on Aug. 10, 2023.

(51) Int. Cl. G01N 33/575; G01N 33/68

(52) U.S. Cl. CPC G01N 33/57557; G01N 33/57585; G01N 33/5759; G01N 33/57595; G01N 33/6863; G01N 33/6872; G01N 2333/523; G01N 2333/70575; G01N 2333/70578

(57) ABSTRACT

Compositions, methods, and kits are provided for diagnosing and treating vestibular schwannoma. In particular, biomarkers associated with tumor growth and hearing loss have been identified that may be useful for diagnosing vestibular schwannoma and aid in determining the timing of tumor resection. These biomarkers can be used alone or in combination with one or more additional biomarkers or relevant clinical parameters in prognosis, diagnosis, or monitoring treatment of vestibular schwannoma.

### CROSS REFERENCE TO RELATED APPLICATIONS

This application claims benefit of U.S. Provisional Patent Application No. 63/383,407, filed Nov. 11, 2022 and U.S. Provisional Patent Application No. 63/518,820, filed Aug. 10, 2023, which applications are incorporated herein by reference in their entireties.

### STATEMENT REGARDING FEDERALLY SPONSORED RESEARCH OR DEVELOPMENT

This invention was made with Government support under contract DC015824 awarded by the National Institutes of Health. The Government has certain rights in the invention.

### BACKGROUND OF THE INVENTION

A vestibular schwannoma (VS) is an intracranial tumor arising from neoplastic Schwann cells in the vestibular branch of the eighth cranial nerve (Mahaley et al., Clinical neurosurgery 36, 347-352 (1990)). VS is primarily caused by mutations in the neurofibromin 2 (NF2) gene disrupting production of merlin, a tumor suppressor. NF2 mutations may be tumor-specific, as in unilateral, sporadic VS, or rare germline mutations leading to bilateral VS and NF2-related schwannomatosis, formerly known as neurofibromatosis type 2 or NF2 (US incidence 1:10,000 vs. 1:33,000, respectively) (Sobel, J. Neuropathol. Exp. Neurol. 52, 106-113 (1993); Fisher et al., Am J Epidemiol 180, 58-67 (2014); Plotkin et al., Genetics in Medicine: Official Journal of the American College of Genetics 24, 1967-1977 (2022)). The most common symptoms of VS are related to disturbed cochlear nerve function (95%) and manifest as sensorineural hearing loss (Genther et al., Jama 309, 2322-2324 (2013)), tinnitus (60% of patients), followed by balance dysfunction (Genther et al., supra; Matthies et al., Neurosurgery 40, 1-9; discussion 9-10 (1997)). Although histologically non-malignant, VS can cause substantial morbidity due to where it typically arises within the internal auditory canal, and can be life-threatening if unchecked expansion into the cerebellopontine angle compresses the brainstem (Mohammadi et al., Otol Neurotol 37, 564-567 (2016)).

VS management is informed by the rate of tumor growth, which is monitored with clinical imaging (i.e., magnetic resonance imaging [MRI]) (Smouha et al., Laryngoscope 115, 450-454 (2005)). Growing tumors are typically treated with microsurgical resection or stereotactic radiation therapy. However, the size and location of sporadic VS do not necessarily correlate with the severity of the tumor-associated hearing loss (Brown et al., Frontiers in oncology 12, 836504 (2022)), thus, the “wait and scan strategy” may be insufficient to determine the ideal timing for tumor resection to prevent progressive hearing loss (Akpinar et al., Int J Radiat Oncol Biol Phys 95, 729-734 (2016); Sughrue et al., J Neurosurg 114, 381-385 (2011); and Sughrue et al., J Neurosurg 112, 163-167 (2010)).

### SUMMARY OF THE INVENTION

Compositions, methods, and kits are provided for diagnosing and treating vestibular schwannoma. In particular, biomarkers associated with tumor growth and hearing loss have been identified that may be useful for diagnosing vestibular schwannoma and aid in determining the timing of tumor resection. These biomarkers can be used alone or in combination with one or more additional biomarkers or relevant clinical parameters in prognosis, diagnosis, or monitoring treatment of vestibular schwannoma.

In one aspect, a method of diagnosing and treating vestibular schwannoma in a patient is provided, the method comprising: obtaining a biological sample from the patient; measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample, wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma; wherein an increased level of eotaxin in the biological sample compared to a reference value range for the level of eotaxin, if the patient is male, indicates that the patient has vestibular schwannoma; wherein an increased level of MCP-3 in the biological sample compared to a reference value range for the level of MCP-3 indicates that the patient has serviceable hearing and word recognition, and wherein a decreased level of MCP-3 in the biological sample compared to the reference value range for the level of MCP-3 indicates that the patient has loss of hearing and word recognition; wherein an increased level of S100B in the biological sample compared to a reference value range for the level of S100B correlates with increased vestibular schwannoma tumor volume; and treating the patient for the vestibular schwannoma if the patient has a positive diagnosis for vestibular schwannoma.

In certain embodiments, the vestibular schwannoma is sporadic vestibular schwannoma.

In certain embodiments, the biological sample is blood or plasma.

In certain embodiments, the levels of at least 2, at least 3, at least 4, at least 5, at least 6, at least 7, at least 8, or at least 9 of the biomarkers are measured in the biological sample.

In certain embodiments, the levels of TNF-R2, MIF, CD30, MCP-3, IL-2R, BLC, TWEAK, eotaxin, and S100B are measured in the biological sample.

In certain embodiments, the levels of TNF-R2, MCP-3, and S100B are measured in the biological sample.

In certain embodiments, the method further comprises measuring levels of monocyte chemoattractant protein-2 (MCP-2) and stromal cell-derived factor 1a (SDF-1a) if the patient is male, wherein increased levels of MCP-2 and SDF-1a in the biological sample compared to reference value ranges for the levels of the MCP-2 and SDF-1a in combination with increased levels of the one or more biomarkers selected from MCP-3, CD30, IL-2R, TWEAK, TNF-R2, S100B, MIF, BLC, and eotaxin in the biological sample compared to the reference value ranges for the levels of the one or more biomarkers selected from MCP-3, CD30, IL-2R, TWEAK, TNF-R2, S100B, MIF, BLC, and eotaxin indicate that the patient has vestibular schwannoma.

In certain embodiments, the method further comprises measuring a level of A proliferation-inducing ligand (APRIL) if the patient is female, wherein an increased level of APRIL in the biological sample compared to a reference value range for the level of APRIL in combination with increased levels of the one or more biomarkers selected from MCP-3, CD30, IL-2R, TWEAK, TNF-R2, S100B, MIF, and BLC in the biological sample compared to the reference value ranges for the levels of the one or more biomarkers selected from MCP-3, CD30, IL-2R, TWEAK, TNF-R2, S100B, MIF, and BLC indicate that the patient has vestibular schwannoma.

In certain embodiments, treating the patient for the vestibular schwannoma comprises microsurgical resection of the vestibular schwannoma tumor, radiosurgery, stereotactic radiation therapy, or a combination thereof. In some embodiments, the surgical resection is performed using a ranslabyrinthine, retrosigmoid, or middle cranial fossa incision.

In certain embodiments, the method further comprises performing medical imaging of the vestibular schwannoma tumor if the level of S100B indicates that the patient has a vestibular schwannoma tumor with a volume large enough to affect hearing function or damage the cochlear nerve. In certain embodiments, the method further comprises performing the microsurgical resection of the vestibular schwannoma tumor if medical imaging and the level of S100B indicates that the patient has a vestibular schwannoma tumor with a volume large enough to affect hearing function or damage the cochlear nerve.

In certain embodiments, the method further comprises performing medical imaging of the vestibular schwannoma tumor if the level of MCP-3 indicates that the patient has loss of hearing and word recognition.

In certain embodiments, the method further comprises performing a hearing test on the patient if the level of MCP-3 indicates that the patient has loss of hearing and word recognition.

In certain embodiments, the method further comprises performing the microsurgical resection of the vestibular schwannoma tumor if the level of MCP-3 indicates that the patient has loss of hearing and word recognition.

In certain embodiments, the levels of the biomarkers are measured by performing an electrochemiluminescence-based immunoassay, an enzyme-linked immunosorbent assay (ELISA), a radioimmunoassay (RIA), an immunofluorescent assay (IFA), immunohistochemistry, a Western Blot, an aptamer-based proteomic assay, mass spectrometry, liquid chromatography-tandem mass spectrometry, tandem mass spectrometry, an enzymatic or biochemical assay, liquid chromatography, or nuclear magnetic resonance (NMR).

In certain embodiments, the biomarkers are measured using a multiplexed immunoassay.

In certain embodiments, the subject has not yet developed clinical symptoms. In other embodiments, the subject has developed clinical symptoms.

In another aspect, a method of monitoring hearing loss in a patient who has vestibular schwannoma is provided, the method comprising: obtaining a first biological sample from the patient at a first time point and a second biological sample from the patient later at a second time point; measuring levels of MCP-3 in the first biological sample and the second biological sample, wherein detection of a decreased level of the MCP-3 in the second biological sample compared to the first biological sample indicate that the patient's hearing and word recognition are worsening, and wherein detection of an increased level of the MCP3 in the biological sample compared to the first biological sample indicate that the patient's hearing and word recognition are improving; and testing the patient's hearing and word recognition if the level of the MCP3 in the second biological sample indicates that the patient's hearing and word recognition are worsening. In some embodiments, the method further comprises performing medical imaging of the patient's vestibular schwannoma tumor if the level of the MCP3 in the second biological sample indicates the patient's hearing and word recognition are worsening. In some embodiments, the method further comprises performing microsurgical resection of the vestibular schwannoma tumor, radiosurgery, stereotactic radiation therapy, or a combination thereof if the patient's hearing and word recognition are worsening.

In another aspect, a method of monitoring tumor volume in a patient who has vestibular schwannoma is provided, the method comprising: obtaining a first biological sample from the patient at a first time point and a second biological sample from the patient later at a second time point; measuring levels of S100B in the first biological sample and the second biological sample, wherein detection of an increased level of the S100B in the second biological sample compared to the first biological sample indicate that the tumor volume is increasing, and wherein detection of a decreased level of the S100B in the biological sample compared to the first biological sample indicate that the tumor volume is not increasing; and performing medical imaging of the vestibular schwannoma tumor if the level of the S100B in the second biological sample indicates that the patient's vestibular schwannoma tumor volume is increasing. In some embodiments, the method further comprises performing microsurgical resection of the vestibular schwannoma tumor, radiosurgery, stereotactic radiation therapy, or a combination thereof if the level of the S100B and the medical imaging indicate that the patient's tumor volume is increasing.

In another aspect, a kit for diagnosing vestibular schwannoma is provided, the kit comprising instructions for determining whether a subject has vestibular schwannoma and agents for detecting at least 3 biomarkers selected from the group consisting of TNF-R2, MIF, CD30, MCP-3, IL-2R, BLC, TWEAK, eotaxin, and S100B.

In certain embodiments, the kit comprises agents for detecting TNF-R2, MCP-3, and S100B. In some embodiments, the kit further comprises agents for detecting MIF, CD30, IL-2R, BLC, TWEAK, and eotaxin.

In certain embodiments, the kit further comprises reagents for performing an immunoassay such as, but not limited to, an electrochemiluminescence-based immunoassay, an enzyme-linked immunosorbent assay (ELISA), a radioimmunoassay (RIA), or an immunofluorescent assay (IFA).

In certain embodiments, the kit comprises an aptamer or antibody that specifically binds to TNF-R2, an aptamer or antibody that specifically binds to MCP-3, and an aptamer or antibody that specifically binds to S100B. In some embodiments, the kit further comprises an aptamer or antibody that specifically binds to MIF, an aptamer or antibody that specifically binds to CD30, an aptamer or antibody that specifically binds to IL-2R, an aptamer or antibody that specifically binds to BLC, an aptamer or antibody that specifically binds to TWEAK, and an aptamer or antibody that specifically binds to eotaxin.

In another aspect, a protein selected from the group consisting of tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) for use as a biomarker in diagnosing vestibular schwannoma is provided.

In another aspect, an in vitro method of diagnosing vestibular schwannoma is provided, the method comprising: obtaining a biological sample from the patient; measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample, wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma; wherein an increased level of eotaxin in the biological sample compared to a reference value range for the level of eotaxin, if the patient is male, indicates that the patient has vestibular schwannoma; wherein an increased level of MCP-3 in the biological sample compared to a reference value range for the level of MCP-3 correlates with serviceable hearing and word recognition, and wherein a decreased level of MCP-3 in the biological sample compared to the reference value range for the level of MCP-3 correlates with loss of hearing and word recognition; and wherein an increased level of S100B in the biological sample compared to a reference value range for the level of S100B correlates with increased vestibular schwannoma tumor volume.

### BRIEF DESCRIPTION OF THE DRAWINGS

FIGS. 1A-1D. Demographic and clinical characteristics of the VS patients and controls. (FIGS. 1A, 1C) Discovery cohort; (FIGS. 1B, 1D) Validation cohort; NA, not available; PTA, pure-tone average; VS, vestibular schwannoma; VS-GH, VS patients with good hearing; VS-PH, VS patients with poor hearing; WR, word recognition. *P<0.05, **P<0.01, ***P<0.001. Note: VS-NA refers to the small group of VS-PH patients in the discovery cohort who did not have quantitative audiograms available (n=4).

FIGS. 2A-2D. Comparison of candidate biomarker plasma levels between VS patients and controls in the discovery and validation cohorts. (FIG. 2A) The concentrations of 14 candidate biomarkers significantly differed between VS patients and controls (discovery cohort). (FIG. 2B) Thirteen of 14 candidate biomarkers were confirmed significantly different in the validation cohort. *Padj<0.05, **Padj<0.01, ***Padj<0.001. Ratios of candidate biomarker levels between VS patients and controls in the discovery (FIG. 2C) and validation (FIG. 2D) cohorts. The full names of candidate biomarkers are listed in table S1. Padj, adjusted P value corrected for multiple comparisons.

FIGS. 3A-3C. MCP-3 is associated with preoperative word recognition score and is elevated in VS patients with SH. The discovery cohort (FIG. 3A) and validation cohort-A (FIG. 3B) were composed of 78 and 50 VS patients, respectively. Validation cohort-B (FIG. 3C) was created by including additional 13 randomly selected patients out of 78 VS patients from the discovery cohort to reach a balanced number of patients in both groups. SH was defined as AAO-HNS class A and B hearing (PTA≤50 dB and WR score≥50%). NSH was defined as AAO-HNS class C and D hearing (either PTA>50 dB or WRS<50%). AAO-HNS, American Academy of Otolaryngology-Head and Neck Surgery; FDR, false discovery rate; #, statistically significant at the FDR of 0.10. Gray, patients of both sexes; red, female sex; blue, male sex.

FIGS. 4A-4C. S100B is associated with preoperative tumor volume and is elevated in VS patients who underwent subtotal tumor resection. The discovery cohort (FIG. 4A) and validation cohort-A (FIG. 4B) were composed of 122 and 50 VS patients, respectively. Validation cohort-B (FIG. 4C) was created by augmenting validation cohort-A with 36 randomly selected patients out of 122 VS patients from the discovery cohort to reach a balanced number of patients in both groups (86 patients per group). GTR, gross tumor resection; STR, subtotal tumor resection; #, statistically significant at the FDR of 0.10. Gray, patients of both sexes; red, female sex; blue, male sex.

FIGS. 5A-5B. Diagnostic utility and discriminatory power of candidate biomarkers with significant AUC values. ROC analysis was performed in the discovery (FIG. 5A) and validation cohorts (FIG. 5B). AUC, area under the curve; SE, sensitivity; SP, specificity; CR, criterion.

FIG. 6A-6D. Tenfold cross-validation and permutation tests of the panel of nine candidate biomarkers selected as the best predictors associated with hearing and tumor size in VS patients. Tenfold cross-validation and permutation tests were performed in the discovery (FIGS. 6A and 6C, respectively) and validation cohorts (FIGS. 6B and 6D, respectively). The nine biomarkers in the panel (DII) were TNR-R2, MIF, CD30, MCP-3, IL-2R, BLC, TWEAK, eotaxin, and S100B. DII, biomarker combination 502 of 502; CV, cross-validation; Opt, optimal.

FIGS. 7A-7B. CONSORT flow diagram for the VS cohort. Sample selection flowchart for the VS discovery (FIG. 7A) and validation cohorts (FIG. 7B). Among the overall cohort of eligible VS patients, subgroups were further defined by pre-operative hearing ability based on AAO-HNS guidelines. Notes: ^{a}Eligible patients had unilateral, sporadic VS that had not been previously resected or irradiated; ^{b}Four VS patients had no pre-operative audiograms (discovery cohort) and five VS patients had missing pure tone average or word recognition scores (validation cohort). ^{c}Serviceable hearing was defined as AAO-HNS Class A and B hearing (PTA≤50 dB and WR score≥50%). ^{d}Non-serviceable hearing was defined as AAO-HNS Class C and D hearing (either PTA>50 dB or WRS<50%). ^{e}GH was defined as word recognition score>70% and pure tone average <30 decibels (dB) (AAO-HNS Class A). ^{f}PH was defined as word recognition score ≤70% and pure tone average ≥30 dB (AAO-HNS Class B, C and D). Abbreviation: NF2, neurofibromatosis 2.

FIGS. 8A-8B. Hearing characteristics of VS patients in the discovery (FIG. 8A) and validation (FIG. 8B) cohorts. Abbreviation: dB, decibel; GH, good hearing; Hz, hertz; PH, poor hearing; VS. vestibular schwannoma; WR, word recognition.

FIG. 9. Significantly elevated candidate biomarkers in female and male VS patients. Abbreviations: VS, vestibular schwannoma. *Padj<0.05, **Padj<0.01, ***Padj<0.001, #significant at P<0.05 prior to adjustment.

FIG. 10. Plasma levels of candidate biomarkers in VS-GH patients, VS-PH patients, and controls. Abbreviations: GH, good hearing; PH, poor hearing; VS, vestibular schwannoma. *Padj<0.05, **Padj<0.01, ***Padj<0.001, #significant at P<0.05 prior to adjustment.

FIGS. 11A-11B. Significantly elevated candidate biomarkers in female and male VS patients with (FIG. 11A) poor or (FIG. 11B) good hearing compared to controls. Abbreviations: GH, good hearing; PH, poor hearing: VS, vestibular schwannoma. *Padj<0.05, **Padj<0.01, ***Padj<0.001, #significant at P<0.05 prior to adjustment.

FIG. 12. Plasma levels of candidate biomarkers in VS patients with serviceable and non-serviceable hearing. The serviceable hearing was defined as AAO-HNS Class A and B hearing (PTA ≤50 dB and WRS >50%). Non-serviceable hearing was defines as AAO-HNS Class C and D hearing (either PTA>50 dB or WRS <50%). Elevated MCP-3 is associated with serviceable hearing. IL-16 and S100B showed a trend for association with serviceable hearing. *Padj<0.05, # and ##significant at P<0.05 and P<0.001, respectively prior to adjustment. The full names of candidate biomarkers are listed in Table S1. Abbreviations: SH, serviceable hearing; NSH, non-serviceable hearing.

FIG. 13. Correlation networks of candidate biomarkers. Abbreviation: VS, vestibular schwannoma.

### DETAILED DESCRIPTION OF THE INVENTION

Compositions, methods, and kits are provided for diagnosing and treating vestibular schwannoma. In particular, biomarkers associated with tumor growth and hearing loss have been identified that may be useful for diagnosing vestibular schwannoma and aid in determining the timing of tumor resection. These biomarkers can be used alone or in combination with one or more additional biomarkers or relevant clinical parameters in prognosis, diagnosis, or monitoring treatment of vestibular schwannoma.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”. “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g., peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

Biomarkers. The term “biomarker” as used herein refers to a compound, such as a protein, a mRNA, a metabolite, or a metabolic byproduct which is differentially expressed or present at different concentrations, levels or frequencies in one sample compared to another, such as a biological sample (e.g., blood or plasma) from patients who have vestibular schwannoma compared to a biological sample from healthy control subjects (i.e., subjects not having vestibular schwannoma). Biomarkers include, but are not limited to, tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, S100 calcium binding protein B (S100B), monocyte chemoattractant protein-2 (MCP-2), stromal cell-derived factor 1a (SDF-1a), and A proliferation-inducing ligand (APRIL).

In some embodiments, the concentration or level of a biomarker is determined before and after the administration of a treatment to a patient. The treatment may comprise, for example, without limitation, microsurgical resection of the vestibular schwannoma tumor, radiosurgery, or stereotactic radiation therapy. The degree of change in the concentration or level of a biomarker, or lack thereof, is interpreted as an indication of whether the treatment has the desired effect (e.g., eradicating tumor or reducing tumor volume and/or preventing or reducing loss of hearing). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., aptamer-based assays, immunoassays (e.g., ELISA, electrochemiluminescence-based immunoassay, or immunofluorescent assay), mass spectrometry (e.g., LC-MS, GC-MS, or tandem mass spectrometry), NMR, biochemical or enzymatic assays, PCR, microarray analysis, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “vestibular schwannoma” biomarker expression profile, a “sporadic vestibular schwannoma” biomarker expression profile, a “vestibular schwannoma with loss of hearing and word recognition” biomarker expression profile, a “vestibular schwannoma with serviceable hearing” biomarker expression profile, or a “vestibular schwannoma tumor with a certain volume” biomarker expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

The term “biological sample” encompasses samples of fluid or tissue isolated from a subject, including but not limited to, blood, plasma, serum, fecal matter, urine, bone marrow, bile, cerebral spinal fluid, lymph fluid, fine needle aspirate, cystic aspirate, a paracentesis sample, a thoracentesis sample, samples of the skin, external secretions of the skin, respiratory, intestinal, and genitourinary tracts, tears, saliva, milk, blood cells, organs, tissue obtained by surgical resection or biopsy, and also samples of in vitro cell culture constituents, including but not limited to, cells in culture, cell lysates, conditioned media resulting from the growth of cells and tissues in culture medium, recombinant cells, and cell components. The definition also includes samples that have been manipulated in any way after their procurement, such as by treatment with reagents, washed, or enriched for particular types of molecules, e.g., biomarker proteins, peptides, etc.

Obtaining and assaying a sample. The term “assaying” is used herein to include the physical steps of manipulating a biological sample to generate data related to the biological sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples will be known to one of ordinary skill in the art and any convenient method may be used to isolate a biological sample.

It will be understood by one of ordinary skill in the art that in some cases, it is convenient to wait until multiple samples have been obtained prior to assaying the samples. Accordingly, in some cases an isolated biological sample is stored until all appropriate samples have been obtained. One of ordinary skill in the art will understand how to appropriately store a variety of different types of biological samples and any convenient method of storage may be used (e.g., refrigeration) that is appropriate for the particular biological sample. In some embodiments, a pre-treatment biological sample is assayed prior to obtaining a post-treatment biological sample. In some cases, a pre-treatment biological sample and a post-treatment biological sample are assayed in parallel. In some cases, multiple different post-treatment biological samples and/or a pre-treatment biological sample are assayed in parallel. In some cases, biological samples are processed immediately or as soon as possible after they are obtained.

In some embodiments, the concentration (i.e., “level”), or expression level of a gene product, which may be a protein, peptide, etc., (which will be referenced herein as a biomarker), in a biological sample is measured (i.e., “determined”). By “expression level” (or “level”) it is meant the level of gene product (e.g., the absolute and/or normalized value determined for the RNA expression level of a biomarker or for the expression level of the encoded polypeptide, or the concentration of the protein in a biological sample). The term “gene product” or “expression product” are used herein to refer to the RNA transcription products (RNA transcripts, e.g., mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple biological samples from the same individual (e.g., biological samples taken at different points in time from the same individual). This is because the units cancel when calculating a fold-change (i.e., determining a ratio) in the expression level from one biological sample to the next (e.g., biological samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

For measuring protein levels, the amount or level of a protein in the biological sample is determined. In some cases, the protein comprises a post-translational modification (e.g., phosphorylation, glycosylation) associated with regulation of activity of the protein such as by a signaling cascade, wherein the modified protein is the biomarker, and the amount of the modified protein is therefore measured. In some embodiments, an extracellular protein level is measured. For example, in some cases, the protein (i.e., polypeptide) being measured is a secreted protein, and the concentration can be measured in aqueous humor. In some embodiments, concentration is a relative value measured by comparing the level of one protein relative to another protein. In other embodiments the concentration is an absolute measurement of weight/volume or weight/weight.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different methods of assaying protein levels are known to one of ordinary skill in the art, and any convenient method may be used, two representative and convenient techniques for assaying protein levels include antibody-based methods such as the enzyme-linked immunosorbent assay (ELISA) and electrochemiluminescence-based immunoassays.

In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Electrochemiluminescence-based immunoassays utilize a biomarker-specific antibody tagged with an electrochemiluminescent luminophore that generates a high-energy species in an electron transfer reaction at an electrode. The species generated at the electrode is in an electronically excited state, which emits light upon relaxation to a lower-energy state. Photons generated by the electrochemiluminescent luminophore may be detected, for example, with photomultiplier tubes or silicon photodiode or gold coated fiber-optic sensors. In some embodiments, the electrochemiluminescent luminophore comprises a ruthenium complex (e.g., Ru(bpy)₃²⁺) or silicon nanoparticle. For a description of electrochemiluminescence-based immunoassays, see, e.g., Wang et al. (2023) Bioelectrochemistry 149:108281, Muzyka et al. (2014) Biosens. Bioelectron. 54:393-407, Keustermans et al. (2013) Methods 61 (1): 10⁻⁷, and Sornambigai et al. (2023) Anal. Bioanal. Chem. 415 (24): 5875-5898; herein incorporated by reference in their entireties.

Alternatively, other methods for measuring the levels of one or more proteins in a sample may be employed. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, and the like) as well as non-antibody-based methods (e.g., aptamer-based assays, nuclear magnetic resonance, mass spectrometry, liquid chromatography-mass spectrometry, or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### Additional Terms.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with vestibular schwannoma) as well as those in which prevention is desired, those with a genetic predisposition to developing vestibular schwannoma, those with increased susceptibility to vestibular schwannoma, those suspected of having vestibular schwannoma, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The terms “individual”, “subject”, and “patient”, are used interchangeably herein and refer to any mammalian subject for whom diagnosis, treatment, or therapy is desired, particularly humans. Mammals include human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows. In some cases, the methods of the invention find use in experimental animals, in veterinary application, and in the development of animal models for disease, including, but not limited to, rodents including mice, rats, and hamsters; primates, and transgenic animals.

“Substantially purified” generally refers to isolation of a component such as a substance (compound, drug, inhibitor, metabolite, nucleic acid, polynucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The terms “polypeptide,” “peptide” and “protein” are used interchangeably herein to refer to a polymer of amino acid residues. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BITE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of biological sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have vestibular schwannoma, the predicted tumor volume, risk of loss of hearing and word recognition, a recommendation for additional screening for pathology, a recommendation for treatment, and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Biomarkers and Diagnostic Methods

Biomarkers that can be used in the practice of the subject methods for diagnosing vestibular schwannoma include, without limitation, tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, S100 calcium binding protein B (S100B), monocyte chemoattractant protein-2 (MCP-2), stromal cell-derived factor 1a (SDF-1a), and A proliferation-inducing ligand (APRIL).

In certain embodiments, a panel of biomarkers is provided. Biomarker panels of any size can be used in the practice of the subject methods. Biomarker panels typically comprise at least 3 biomarkers and up to 20 biomarkers, including any number of biomarkers in between, such as 3, 4, 5, 6, 7, 8, 9, 10, 11, 12, 13, 14, 15, 16, 17, 18, 19, or 20 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20, or more biomarkers. Although smaller biomarker panels are usually more economical, larger biomarker panels (i.e., greater than 20 biomarkers) have the advantage of providing more detailed information and can also be used in the practice of the subject methods.

In some embodiments, a biomarker panel for diagnosing vestibular schwannoma comprises or consists of TNF-R2, MIF, CD30, MCP-3, IL-2R, BLC, TWEAK, eotaxin, and S100B.

In some embodiments, a biomarker panel for diagnosing vestibular schwannoma comprises or consists of TNF-R2, MCP-3, and S100B.

In some embodiments, a biomarker panel for diagnosing vestibular schwannoma comprises a set of biomarkers listed in Table S3 of Example 1.

The biological sample obtained from the subject to be diagnosed is typically blood or plasma, but can be any sample from bodily fluids, tissue or cells that contain the expressed biomarkers. A “control” sample as used herein refers to a biological sample, such as blood, plasma, tissue, or cells that are not diseased. That is, a control sample is obtained from a normal subject (e.g., an individual known to not have vestibular schwannoma or any condition or symptom associated with the disease). A biological sample can be obtained from a subject by conventional techniques. For example, blood can be obtained by venipuncture, while plasma and serum can be obtained by fractionating whole blood according to known methods. Surgical techniques for obtaining solid tissue samples are well known in the art.

When analyzing the levels of biomarkers in a biological sample from a subject, the reference value ranges used for comparison can represent the levels of one or more biomarkers in a biological sample from one or more subjects without disease (i.e., normal or healthy control). Alternatively, the reference values can represent the levels of one or more biomarkers from one or more subjects with a disease (e.g., vestibular schwannoma), wherein similarity to the reference value ranges indicates the subject has the disease. More specifically, the reference value ranges can represent the levels of one or more biomarkers from one or more subjects with “vestibular schwannoma (a “vestibular schwannoma” biomarker expression profile), sporadic vestibular schwannoma (a “sporadic vestibular schwannoma” biomarker expression profile), different degrees of hearing loss or diminished word recognition caused by vestibular schwannoma (a “vestibular schwannoma with loss of hearing and word recognition” biomarker expression profile or a “vestibular schwannoma with serviceable hearing” biomarker expression profile), or different size vestibular schwannoma tumors (a “vestibular schwannoma tumor with a certain volume” biomarker expression profile).

Accordingly, in one aspect, a method of diagnosing vestibular schwannoma in a patient is provided, the method comprising: obtaining a biological sample (e.g., blood or plasma) from the patient; measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample, wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma; wherein an increased level of eotaxin in the biological sample compared to a reference value range for the level of eotaxin if the patient is male indicate that the patient has vestibular schwannoma; wherein an increased level of MCP-3 in the biological sample compared to a reference value range for the level of MCP-3 indicates that the patient has serviceable hearing and word recognition, and wherein a decreased level of MCP-3 in the biological sample compared to the reference value range for the level of MCP-3 indicates loss of hearing and word recognition in the patient; wherein an increased level of S100B in the biological sample compared to a reference value range for the level of S100B correlates with increased vestibular schwannoma tumor volume; and treating the patient for the vestibular schwannoma if the patient has a positive diagnosis for vestibular schwannoma. In certain embodiments, the levels of TNF-R2, MIF, CD30, MCP-3, IL-2R, BLC, TWEAK, eotaxin, and S100B are measured in the biological sample. In certain embodiments, the levels of TNF-R2, MCP-3, and S100B are measured in the biological sample.

In certain embodiments, the method further comprises measuring levels of monocyte chemoattractant protein-2 (MCP-2) and SDF-1a if the patient is male, wherein increased levels of MCP-2 and SDF-1a in the biological sample compared to reference value ranges for the levels of MCP-2 and SDF-1a in combination with increased levels of the one or more biomarkers selected from MCP-3, CD30, IL-2R, TWEAK, TNF-R2, S100B, MIF, BLC, and eotaxin in the biological sample compared to the reference value ranges for the levels of the one or more biomarkers selected from MCP-3, CD30, IL-2R, TWEAK, TNF-R2. S100B, MIF, BLC, and eotaxin indicate that the patient has vestibular schwannoma.

In certain embodiments, the method further comprises measuring a level of A proliferation-inducing ligand (APRIL) if the patient is female, wherein an increased level of APRIL in the biological sample compared to a reference value range for the level of APRIL in combination with increased levels of the one or more biomarkers selected from MCP-3, CD30, IL-2R, TWEAK, TNF-R2, S100B, MIF, and BLC in the biological sample compared to the reference value ranges for the levels of the one or more biomarkers selected from MCP-3, CD30, IL-2R, TWEAK, TNF-R2, S100B, MIF, and BLC indicate that the patient has vestibular schwannoma.

The method may further comprise determining an appropriate treatment regimen for a patient and, in particular, whether a patient should be treated for vestibular schwannoma. For example, a patient is selected for treatment for vestibular schwannoma if the patient has a positive diagnosis for vestibular schwannoma based on a biomarker expression profile, as described herein. The treatment for vestibular schwannoma may comprise, for example, microsurgical resection of the vestibular schwannoma tumor, radiosurgery, stereotactic radiation therapy, or a combination thereof. In some embodiments, the surgical resection is performed using a ranslabyrinthine, retrosigmoid, or middle cranial fossa incision.

In certain embodiments, a patient who has vestibular schwannoma is monitored for hearing loss by a method comprising: obtaining a first biological sample from the patient at a first time point and a second biological sample from the patient later at a second time point; measuring levels of MCP-3 in the first biological sample and the second biological sample, wherein detection of a decreased level of the MCP-3 in the second biological sample compared to the first biological sample indicate that the patient's hearing and word recognition are worsening, and wherein detection of an increased level of the MCP3 in the biological sample compared to the first biological sample indicate that the patient's hearing and word recognition are improving; and testing the patient's hearing and word recognition if the level of the MCP3 in the second biological sample indicates that the patient's hearing and word recognition are worsening. In some embodiments, the method further comprises performing medical imaging of the patient's vestibular schwannoma tumor if the level of the MCP3 in the second biological sample indicates the patient's hearing and word recognition are worsening. In certain embodiments, the method further comprises performing the microsurgical resection of the vestibular schwannoma tumor if the level of MCP-3 indicates that the patient has loss of hearing and word recognition.

In certain embodiments, a patient who has vestibular schwannoma is monitored for changes in tumor volume by a method comprising: obtaining a first biological sample from the patient at a first time point and a second biological sample from the patient later at a second time point; measuring levels of S100B in the first biological sample and the second biological sample, wherein detection of an increased level of the S100B in the second biological sample compared to the first biological sample indicate that the tumor volume is increasing, and wherein detection of a decreased level of the S100B in the biological sample compared to the first biological sample indicate that the tumor volume is not increasing; and performing medical imaging of the vestibular schwannoma tumor if the level of the S100B 3 in the second biological sample indicates that the patient's vestibular schwannoma tumor volume is increasing.

Medical imaging may be used to confirm whether a tumor is shrinking or growing and the extent of the tumor to aid in determining prognosis and evaluating optimal strategies for treatment. In certain embodiments, medical imaging of the tumor is performed, for example, by magnetic resonance imaging (MRI), positron emission tomography (PET), single photon emission computed tomography (SPECT), computed tomography (CT), ultrasound imaging (UI), optical imaging (OI), photoacoustic imaging (PI), fluoroscopy, or fluorescence imaging. In some embodiments, the method further comprises performing microsurgical resection of the vestibular schwannoma tumor, radiosurgery, stereotactic radiation therapy, or a combination thereof if the patient's tumor volume is increasing and/or the patient's hearing and word recognition are worsening.

The subject methods may also be used for assaying pre-treatment and post-treatment biological samples obtained from an individual to determine whether the individual is responsive or not responsive to a treatment. For example, a first biological sample can be obtained from a subject before the subject undergoes the therapy, and a second biological sample can be obtained from the subject after the subject undergoes the therapy. In one embodiment, the efficacy of a treatment of a patient for vestibular schwannoma is monitored by measuring one or more biomarkers selected from TNF-R2, MIF, CD30, MCP-3, IL-2R, BLC, TWEAK, eotaxin, and S100B; and evaluating the efficacy of the treatment, wherein detection of increased levels of the one or more biomarkers selected from TNF-R2, MIF, CD30, MCP-3, IL-2R, BLC, TWEAK, eotaxin, and S100B in the second biological sample compared to the first biological sample indicate that the patient is worsening or not responding to the treatment, and detection of decreased levels of the one or more biomarkers selected from TNF-R2, MIF, CD30, MCP-3, IL-2R, BLC, TWEAK, eotaxin, and S100B in the second biological sample compared to the first biological sample indicate that the patient is improving. In certain embodiments, the method further comprises altering the treatment if the patient is worsening or not responding to the treatment.

The level of a biomarker in a pre-treatment biological sample can be referred to as a “pre-treatment value” because the first biological sample is isolated from the individual prior to the administration of the therapy (i.e., “pre-treatment”). The level of a biomarker in the pre-treatment biological sample can also be referred to as a “baseline value” because this value is the value to which “post-treatment” values are compared. In some cases, the baseline value (i.e., “pre-treatment value”) is determined by determining the level of a biomarker in multiple (i.e., more than one, e.g., two or more, three or more, for or more, five or more, etc.) pre-treatment biological samples. In some cases, the multiple pre-treatment biological samples are isolated from an individual at different time points in order to assess natural fluctuations in biomarker levels prior to treatment. As such, in some cases, one or more (e.g., two or more, three or more, for or more, five or more, etc.) pre-treatment biological samples are isolated from the individual. In some embodiments, all of the pre-treatment biological samples will be the same type of biological sample (e.g., a plasma sample). In some cases, two or more pre-treatment biological samples are pooled prior to determining the level of the biomarker in the biological samples. In some cases, the level of the biomarker is determined separately for two or more pre-treatment biological samples and a “pre-treatment value” is calculated by averaging the separate measurements.

A post-treatment biological sample is isolated from an individual after the administration of a therapy. Thus, the level of a biomarker in a post-treatment sample can be referred to as a “post-treatment value”. In some embodiments, the level of a biomarker is measured in additional post-treatment biological samples (e.g., a second, third, fourth, fifth, etc. post-treatment biological sample). Because additional post-treatment biological samples are isolated from the individual after the administration of a treatment, the levels of a biomarker in the additional biological samples can also be referred to as “post-treatment values.”

The term “responsive” as used herein means that the treatment is having the desired effect such as improving hearing and word recognition, preventing, reducing or delaying loss of hearing and word recognition, and/or preventing or reducing tumor growth. When the individual does not improve in response to the treatment, it may be desirable to seek a different therapy or treatment regime for the individual.

The determination that an individual has vestibular schwannoma or is at risk of hearing loss or tumor growth by expression profiling is an active clinical application of the correlation between levels of a biomarker and vestibular schwannoma, hearing loss, or tumor volume. For example, “determining” requires the active step of reviewing the data, which is produced during the active assaying step(s), and determining whether an individual does or does not have vestibular schwannoma or is at risk of hearing loss or tumor growth. Additionally, in some cases, a decision is made to proceed with a current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating vestibular schwannoma, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated for vestibular schwannoma by microsurgical resection to partially remove a tumor. Then to “continue treatment” would be to continue with this type of treatment. If the current course of treatment is not effective, the treatment may be altered, e.g., switching treatment to completely remove the tumor, or changing to a different type of treatment such as radiosurgery or stereotactic radiation therapy.

In other words, the level of one or more biomarkers may be monitored in order to determine when to continue therapy and/or when to alter therapy. As such, a post-treatment biological sample can be isolated after any of the administrations and the biological sample can be assayed to determine the level of a biomarker. Accordingly, the subject methods can be used to determine whether an individual being treated for vestibular schwannoma is responsive or is maintaining responsiveness to a treatment.

The therapy can be administered to an individual any time after a pre-treatment biological sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less. 2 minutes or less, or 1 minute or less) after a pre-treatment biological sample is isolated (or, when multiple pre-treatment biological samples are isolated, after the final pre-treatment biological sample is isolated).

In some cases, more than one type of therapy may be administered to the individual. For example, a subject who has vestibular schwannoma may be treated with microsurgical resection to partially remove a tumor followed by stereotactic radiation therapy. A subject who has more severe disease or who is at high risk of disease progression, may be treated more aggressively. For example, treatment of a high-risk patient may include microsurgical resection to completely remove a tumor followed by radiosurgery and/or stereotactic radiation therapy.

In some embodiments, the subject methods include providing an analysis (e.g., an oral or written report) having any or all of the following information: identifying information of the subject (name, age, etc.), a description of what type of biological sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have vestibular schwannoma, the predicted tumor volume, risk of loss of hearing and word recognition, a recommendation for additional screening for pathology, a recommendation for treatment, and/or to continue or alter therapy, a recommended strategy for additional therapy, etc. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in diagnosing a patient with vestibular schwannoma, monitoring risk of tumor growth and loss of hearing and word recognition, and determining the appropriate treatment for a subject, as well as monitoring responses of a subject to treatment.

In some embodiments, the amount or level in the sample of one or more proteins/polypeptides encoded by a gene of interest is determined. Any convenient protocol for evaluating protein levels may be employed, wherein the level of one or more proteins in the assayed sample is determined. One representative and convenient technique for assaying protein levels is an antibody-based method such as the enzyme-linked immunosorbent assay (ELISA).

For antibody-based methods of protein level determination, any convenient antibody can be used that specifically binds to the intended biomarker. The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Another antibody-based method is an electrochemiluminescence-based immunoassay, which utilizes antibodies tagged with an electrochemiluminescent luminophore that generates a high-energy species in an electron transfer reaction at an electrode. The species generated at the electrode is in an electronically excited state, which emits light upon relaxation to a lower-energy state. Photons generated by the electrochemiluminescent luminophore may be detected, for example, with photomultiplier tubes or silicon photodiode or gold coated fiber-optic sensors. In some embodiments, the electrochemiluminescent luminophore comprises a ruthenium complex (e.g., Ru(bpy)₃²⁺) or silicon nanoparticle. For a description of electrochemiluminescence-based immunoassays, see, e.g., Wang et al. (2023) Bioelectrochemistry 149:108281, Muzyka et al. (2014) Biosens. Bioelectron. 54:393-407, Keustermans et al. (2013) Methods 61 (1): 10⁻⁷, and Sornambigai et al. (2023) Anal. Bioanal. Chem. 415 (24): 5875-5898; herein incorporated by reference in their entireties.

Alternatively, other methods for measuring the levels of one or more proteins in a sample may be employed, and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; nuclear magnetic resonance, mass spectrometry, liquid chromatography-mass spectrometry, tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

Aptamer-based assays use aptamers comprising single-stranded oligonucleotides that bind specifically to biomarker proteins of interest. Either high affinity RNA or DNA aptamers with specificity for a protein of interest may be used. Functional groups that mimic amino acid side-chains may be added to aptamers to confer protein-like properties to improve binding affinity to a protein of interest. Aptamers that bind specifically and with high affinity to a protein of interest can be selected from large libraries of aptamers having randomized sequences using Systematic Evolution of Ligands by EXponential enrichment (SELEX). The aptamers may be designed with unique nucleotide sequences recognizable by specific hybridization probes for capture on a hybridization array for multiplexed detection of biomarkers (see, e.g., Gold et al. (2010) Aptamer-Based Multiplexed Proteomic Technology for Biomarker Discovery. PLOS ONE 5 (12): e15004; herein incorporated by reference in its entirety.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

In some embodiments, mass spectrometry is used for detection of biomarkers. Mass spectrometry detection of molecules typically involves formation of gas phase ions through electron impact ionization (EI), electrospray ionization (ESI), heated electrospray ionization (HESI), or matrix-assisted laser desorption/ionization (MALDI). In some cases, biomarkers in samples are separated prior to ionization using gas chromatography (GC-MS) or liquid chromatography (LC-MS). For a description of mass spectrometry techniques for identifying biomarkers, see, e.g., Geyer et al. (2017) Mol Syst Biol. 13 (9): 942, Kang et al. (2020) Biomed Chromatogr. 34 (1): e4633, Nakayasu et al. (2021) Nat Protoc. 16 (8): 3737-3760, Chen et al. (2016) Adv. Exp. Med. Biol. 919:255-279, Hawkridge et al. (2009) Annu Rev Anal Chem 2:265-277, Chace (2001) Chem Rev. 101:445-477; herein incorporated by reference in their entireties.

For measuring protein activity levels, the amount or level of protein activity in the sample of one or more proteins/polypeptides encoded by the gene of interest is determined.

In other embodiments, the amount or level in the sample of one or more proteins is determined. Any convenient method for measuring protein levels in a sample may be used, e.g., antibody-based methods, e.g., aptamer-based assays, immunoassay such as enzyme-linked immunosorbent assays (ELISAs), immunohistochemistry, and mass spectrometry.

The resultant data provides information regarding expression, amount, and/or activity for each of the biomarkers that have been measured, wherein the information is in terms of whether or not the biomarker is present (e.g., expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for vestibular schwannoma or hearing loss, or tumor size. In some embodiments, measurements for a biomarker or combinations of biomarkers are formulated into linear or non-linear models or algorithms (e.g., a ‘biomarker signature’) and converted into a likelihood score. This likelihood score indicates the probability that a biological sample is from a patient having vestibular schwannoma, or a patient who may exhibit no evidence of disease, or a patient who may exhibit a particular type of vestibular schwannoma (e.g., sporadic vestibular schwannoma or neurofibromatosis type 2, unilateral or bilateral vestibular schwannoma) or risk of hearing loss or tumor growth. The models and/or algorithms can be provided in machine readable format, and may be used to correlate biomarker levels or a biomarker profile with a disease state, and/or to designate a treatment modality for a patient or class of patients.

In some embodiments, a machine learning algorithm is used to classify a patient as having vestibular schwannoma and/or a risk of hearing loss or tumor growth. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithm may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithm comprises a reinforcement learning algorithm. Examples of reinforcement learning algorithms include, but are not limited to, temporal difference learning, Q-learning and Learning Automata. Alternatively, the machine learning algorithm may comprise Data Pre-processing.

In some embodiments, the machine learning algorithm uses artificial neural networks. In some embodiments, the machine learning algorithm uses a deep learning algorithm, which may include the use of convolutional neural networks, deep neural networks, recurrent neural networks, efficient neural networks, deep residual neural networks, long short-term memory networks, deep belief networks, multilayer perceptrons, or deep reinforcement learning, and the like. See, e.g., Pedrycz et al. Deep Learning: Algorithms and Applications (Studies in Computational Intelligence Book 865, Springer, 2019), Goodfellow et al. Deep Learning (Adaptive Computation and Machine Learning series, The MIT Press, 2016), and Various Deep Learning Algorithms in Computational Intelligence (edited by Oscar Humberto Montiel Ross, Mdpi AG, 2023); herein incorporated by reference in their entireties.

### Kits

Also provided are kits for use in the methods, disclosed herein, for diagnosing vestibular schwannoma and determining risk of hearing loss or tumor growth. The subject kits include agents (e.g., an antibody that specifically binds to a biomarker and/or other assay reagents, and the like) for determining the level of at least one biomarker. In some embodiments, a kit comprises agents for determining the level of a single biomarker, two or more different biomarkers, three or more different biomarkers, four or more different biomarkers, five or more different biomarkers, six or more different biomarkers, seven or more different biomarkers, eight or more different biomarkers, or nine or more different biomarkers. In certain embodiments, the kit comprises reagents for performing an immunoassay (e.g., ELISA, electrochemiluminescence-based immunoassay, or immunofluorescent assay).

In certain embodiments, the kit comprises agents for detecting at least 3 biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B).

In certain embodiments, the kit comprises agents for detecting TNF-R2, MCP-3, and S100B. In some embodiments, the kit further comprises agents for detecting MIF, CD30, IL-2R, BLC, TWEAK, and eotaxin. In some embodiments, the kit further comprises agents for detecting one or more biomarkers selected from monocyte chemoattractant protein-2 (MCP-2), SDF-1a, and APRIL.

In some embodiments, the kit comprises an aptamer or antibody that specifically binds to TNF-R2, an aptamer or antibody that specifically binds to MCP-3, and an aptamer or antibody that specifically binds to S100B. In some embodiments, the kit further comprises an aptamer or antibody that specifically binds to MIF, an aptamer or antibody that specifically binds to CD30, an aptamer or antibody that specifically binds to IL-2R, an aptamer or antibody that specifically binds to BLC, an aptamer or antibody that specifically binds to TWEAK, and an aptamer or antibody that specifically binds to eotaxin.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-33 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of diagnosing and treating vestibular schwannoma in a patient, the method comprising:
obtaining a biological sample from the patient;
measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample,
wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma;
wherein an increased level of eotaxin in the biological sample compared to a reference value range for the level of eotaxin, if the patient, is male, indicates that the patient has vestibular schwannoma;
wherein an increased level of MCP-3 in the biological sample compared to a reference value range for the level of MCP-3 indicates that the patient has serviceable hearing and word recognition, and wherein a decreased level of MCP-3 in the biological sample compared to the reference value range for the level of MCP-3 indicates that the patient has loss of hearing and word recognition;
wherein an increased level of S100B in the biological sample compared to a reference value range for the level of S100B correlates with increased vestibular schwannoma tumor volume; and
treating the patient for the vestibular schwannoma if the patient has a positive diagnosis for vestibular schwannoma.

2. The method of aspect 1, wherein the vestibular schwannoma is sporadic vestibular schwannoma.

3. The method of aspect 1 or 2, wherein the biological sample is blood or plasma.

4. The method of any one of aspects 1-3, wherein the levels of at least 2, at least 3, at least 4, at least 5, at least 6, at least 7, at least 8, or at least 9 of the biomarkers are measured in the biological sample.

5. The method of aspect 4, wherein the levels of TNF-R2, MIF, CD30, MCP-3, IL-2R, BLC, TWEAK, eotaxin, and S100B are measured in the biological sample.

6. The method of aspect 4, wherein the levels of TNF-R2, MCP-3, and S100B are measured in the biological sample.

7. The method of any one of aspects 1-6, further comprising measuring levels of monocyte chemoattractant protein-2 (MCP-2) and stromal cell-derived factor 1a (SDF-1a) if the patient is male, wherein increased levels of MCP-2 and SDF-1a in the biological sample compared to reference value ranges for the levels of MCP-2 and SDF-1a in combination with increased levels of the one or more biomarkers selected from MCP-3, CD30, IL-2R, TWEAK, TNF-R2, S100B, MIF, BLC, and eotaxin in the biological sample compared to the reference value ranges for the levels of the one or more biomarkers selected from MCP-3, CD30, IL-2R, TWEAK, TNF-R2, S100B, MIF, BLC, and eotaxin indicate that the patient has vestibular schwannoma.

8. The method of any one of aspects 1-7, further comprising measuring a level of A proliferation-inducing ligand (APRIL) if the patient is female, wherein an increased level of APRIL in the biological sample compared to a reference value range for the level of APRIL in combination with increased levels of the one or more biomarkers selected from MCP-3, CD30, IL-2R, TWEAK, TNF-R2, S100B, MIF, and BLC in the biological sample compared to the reference value ranges for the levels of the one or more biomarkers selected from MCP-3, CD30, IL-2R, TWEAK, TNF-R2, S100B, MIF, and BLC indicate that the patient has vestibular schwannoma.

9. The method of any one of aspects 1-8, wherein said treating the patient for the vestibular schwannoma comprises microsurgical resection of the vestibular schwannoma tumor, radiosurgery, stereotactic radiation therapy, or a combination thereof.

10. The method of aspect 9, wherein the surgical resection is performed using a ranslabyrinthine, retrosigmoid, or middle cranial fossa incision.

11. The method of any one of aspects 1-10, further comprising performing medical imaging of the vestibular schwannoma tumor if the level of S100B indicates that the patient has the vestibular schwannoma tumor with a volume large enough to affect hearing function or damage the cochlear nerve, or if the level of MCP-3 indicates that the patient has loss of hearing or word recognition.

12. The method of aspect 11, wherein the microsurgical resection of the vestibular schwannoma tumor is performed if the medical imaging and the level of S100B indicates that the patient has a vestibular schwannoma tumor with a volume large enough to affect hearing function or damage the cochlear nerve.

13. The method of any one of aspects 1-12, further comprising performing a hearing test on the patient if the level of MCP-3 indicates that the patient has loss of hearing and word recognition.

14. The method of any one of aspects 9-13, wherein the microsurgical resection of the vestibular schwannoma tumor is performed if the level of MCP-3 indicates that the patient has loss of hearing and word recognition.

15. The method of any one of aspects 1-14, wherein said measuring comprises performing an electrochemiluminescence-based immunoassay, an enzyme-linked immunosorbent assay (ELISA), a radioimmunoassay (RIA), an immunofluorescent assay (IFA), immunohistochemistry, a Western Blot, an aptamer-based proteomic assay, mass spectrometry, liquid chromatography-tandem mass spectrometry, tandem mass spectrometry, an enzymatic or biochemical assay, liquid chromatography, or nuclear magnetic resonance (NMR).

16. The method of any one of aspects 1-15, wherein said measuring comprises performing a multiplex immunoassay.

17. The method of any one of aspects 1-16, wherein the subject has not yet developed clinical symptoms.

18. The method of any one of aspects 1-16, wherein the subject has developed clinical symptoms.

19. A method of monitoring hearing loss in a patient who has vestibular schwannoma, the method comprising:
obtaining a first biological sample from the patient at a first time point and a second biological sample from the patient later at a second time point;
measuring levels of MCP-3 in the first biological sample and the second biological sample, wherein detection of a decreased level of the MCP-3 in the second biological sample compared to the first biological sample indicate that the patient's hearing and word recognition are worsening, and wherein detection of an increased level of the MCP3 in the biological sample compared to the first biological sample indicate that the patient's hearing and word recognition are improving; and
testing the patient's hearing and word recognition if the level of the MCP3 in the second biological sample indicates that the patient's hearing and word recognition are worsening.

20. The method of aspect 19, further comprising performing medical imaging of the patient's vestibular schwannoma tumor if the level of the MCP3 in the second biological sample indicates the patient's hearing and word recognition are worsening.

21. The method of aspect 19 or 20, further comprising performing microsurgical resection of the vestibular schwannoma tumor, radiosurgery, stereotactic radiation therapy, or a combination thereof if the patient's hearing and word recognition are worsening.

22. A method of monitoring tumor volume in a patient who has vestibular schwannoma, the method comprising:
obtaining a first biological sample from the patient at a first time point and a second biological sample from the patient later at a second time point;
measuring levels of S100B in the first biological sample and the second biological sample, wherein detection of an increased level of the S100B in the second biological sample compared to the first biological sample indicate that the tumor volume is increasing, and wherein detection of a decreased level of the S100B in the biological sample compared to the first biological sample indicate that the tumor volume is not increasing; and
performing medical imaging of the vestibular schwannoma tumor if the level of the S100B in the second biological sample indicates that the patient's vestibular schwannoma tumor volume is increasing.

23. The method of aspect 22, further comprising performing microsurgical resection of the vestibular schwannoma tumor, radiosurgery, stereotactic radiation therapy, or a combination thereof if the level of the S100B and the medical imaging indicate that the patient's tumor volume is increasing.

24. A kit for diagnosing vestibular schwannoma comprising instructions for determining whether a subject has vestibular schwannoma and agents for detecting at least 3 biomarkers selected from the group consisting of tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B).

25. The kit of aspect 24, wherein the kit comprises agents for detecting TNF-R2, MIF, CD30, MCP-3, IL-2R, BLC, TWEAK, eotaxin, and S100B.

26. The kit of aspect 24, wherein the kit comprises agents for detecting TNF-R2, MCP-3, and S100B.

27. The kit of any one of aspects 24-26, further comprising agents for detecting one or more biomarkers selected from monocyte chemoattractant protein-2 (MCP-2), SDF-1a, and APRIL.

28. The kit of any one of aspects 24-27, further comprising reagents for performing an immunoassay or aptamer assay.

29. The kit of aspect 28, wherein the immunoassay is an electrochemiluminescence-based immunoassay, an enzyme-linked immunosorbent assay (ELISA), a radioimmunoassay (RIA), or an immunofluorescent assay (IFA).

30. The kit of aspect 28 or 29, wherein the kit comprises an aptamer or antibody that specifically binds to TNF-R2, an aptamer or antibody that specifically binds to MCP-3, and an aptamer or antibody that specifically binds to S100B.

31. The kit of aspect 30, wherein the kit further comprises an aptamer or antibody that specifically binds to MIF, an aptamer or antibody that specifically binds to CD30, an aptamer or antibody that specifically binds to IL-2R, an aptamer or antibody that specifically binds to BLC, an aptamer or antibody that specifically binds to TWEAK, and an aptamer or antibody that specifically binds to eotaxin.

32. A protein selected from the group consisting of tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) for use as a biomarker in diagnosing vestibular schwannoma.

33. An in vitro method of diagnosing vestibular schwannoma, the method comprising:
obtaining a biological sample from the patient;
measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample,
wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma;
wherein an increased level of eotaxin in the biological sample compared to a reference value range for the level of eotaxin, if the patient is male, indicates that the patient has vestibular schwannoma;
wherein an increased level of MCP-3 in the biological sample compared to a reference value range for the level of MCP-3 indicates that the patient has serviceable hearing and word recognition, and wherein a decreased level of MCP-3 in the biological sample compared to the reference value range for the level of MCP-3 indicates loss of hearing and word recognition in the patient; and
wherein an increased level of S100B in the biological sample compared to a reference value range for the level of S100B correlates with increased vestibular schwannoma tumor volume.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Identification of Immune-Related Candidate Biomarkers in Plasma of Patients with Sporadic Vestibular Schwannoma

### Introduction

A vestibular schwannoma (VS) is an intracranial tumor arising from neoplastic Schwann cells in the vestibular branch of the eighth cranial nerve (1). VS is primarily caused by mutations in the neurofibromin 2 (NF2) gene disrupting production of merlin, a tumor suppressor. NF2 mutations may be tumor specific, as in unilateral, sporadic VS, or rare germline mutations leading to bilateral VS and NF2-related schwannomatosis, formerly known as neurofibromatosis type 2 (NF2) (U.S. incidence 1:10,000 versus 1:33,000, respectively) (2-4). The most common symptoms of VS are related to disturbed cochlear nerve function (95%) and manifest as sensorineural hearing loss (5), tinnitus (60% of patients), followed by balance dysfunction (5, 6). Although histologically nonmalignant, VS can cause substantial morbidity due to where it typically arises within the internal auditory canal, and can be life-threatening if unchecked expansion into the cerebellopontine angle compresses the brainstem (7).

VS management is informed by the rate of tumor growth, which is monitored with clinical imaging [i.e., magnetic resonance imaging (MRI)] (8). Growing tumors are typically treated with microsurgical resection or stereotactic radiation therapy. However, the size and location of sporadic VS do not necessarily correlate with the severity of the tumor-associated hearing loss (9); thus, the “wait and scan strategy” may be insufficient to determine the ideal timing for tumor resection to prevent progressive hearing loss (10-12).

The identification of blood biomarkers whose levels correlate with hearing loss severity or tumor size, or are prognostic of clinical outcomes, could have immense value for the management of VS (13). Prior attempts to characterize potential blood biomarkers have focused on NF2-related schwannomatosis rather than the more common sporadic VS, which comprises >90% of cases (2, 3) and had limited sample sizes (<30 patients) (14-16). Therefore, we conducted extensive immune profiling of blood plasma to identify candidate biomarkers of the tumor and related hearing loss in sporadic VS, informed by our previous studies in vitro or among smaller cohorts. For example, matrix metalloproteinase-14 (MMP-14) is the most abundant MMP in VS, with differential expression between tumors associated with poor hearing (PH) versus good hearing (GH) (17). Additionally, sporadic VSs associated with GH secreted high levels of fibroblast growth factor 2 (FGF-2), which had an otoprotective effect in vitro (18). Conversely, higher secreted levels of tumor necrosis factor-α (TNF-α) were associated with worse VS-induced hearing loss and had an ototoxic effect in vitro (19). Finally, interleukin-18 (IL-18) levels were significantly elevated in the tumors of VS-PH patients compared to VS-GH patients (20).

Motivated by these data, we quantified the levels of FGF-2, IL-18, and TNF-α in the plasma of >170 patients with sporadic VS and extended the analysis to 66 cytokines, chemokines, growth factors, and cell surface proteins (table S1). We identified and analyzed candidate biomarkers with differential concentrations in the plasma of patients with and without VS (controls) and examined their association with preoperative hearing and tumor volume. Finally, we assessed the diagnostic utility of the candidate biomarkers and a composite biomarker panel in discriminating between VS patients and controls.

### Results

### Patient Characteristics

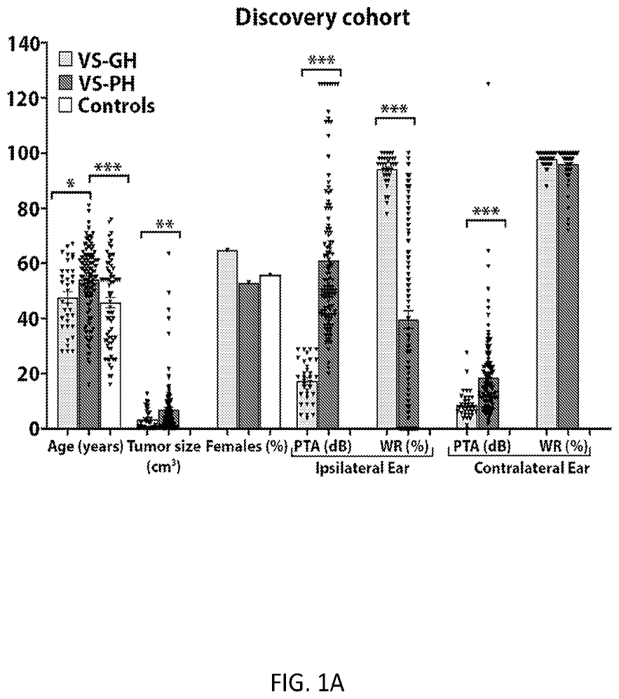

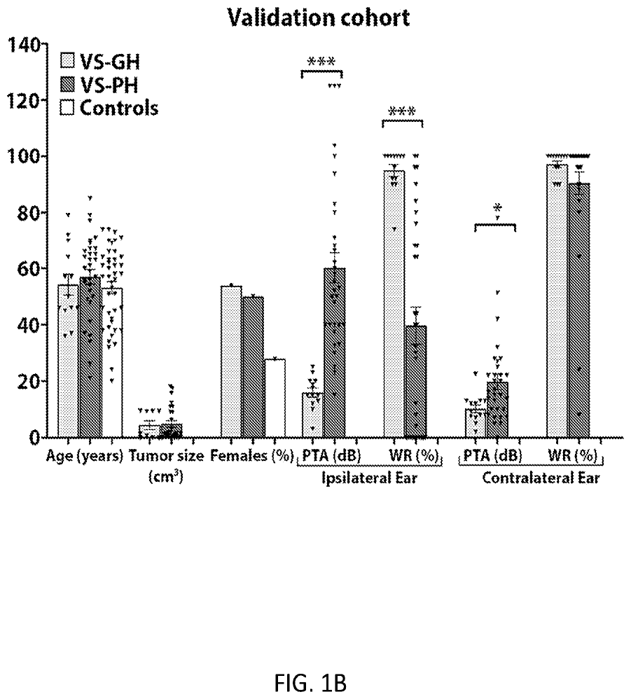

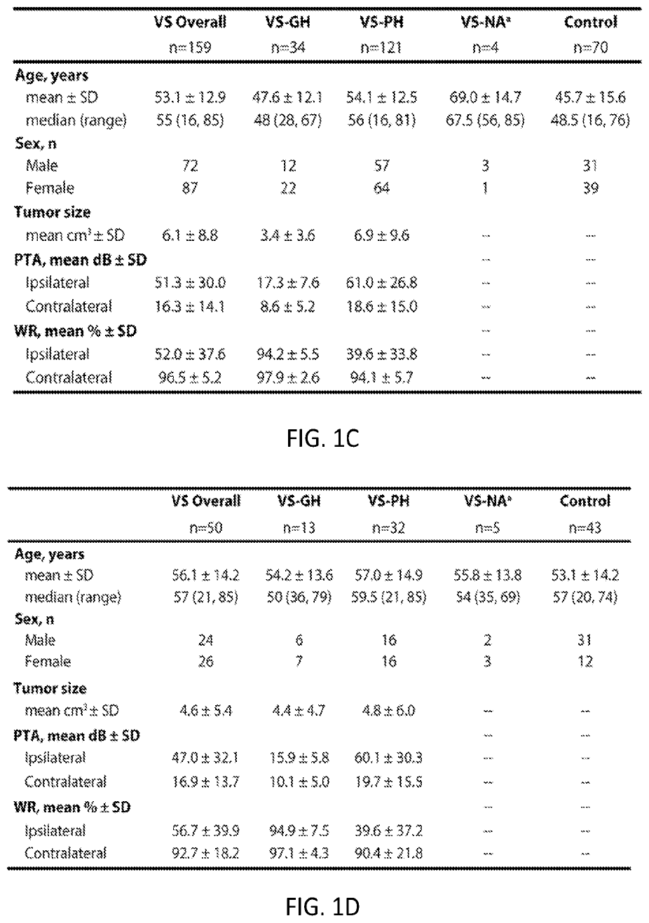

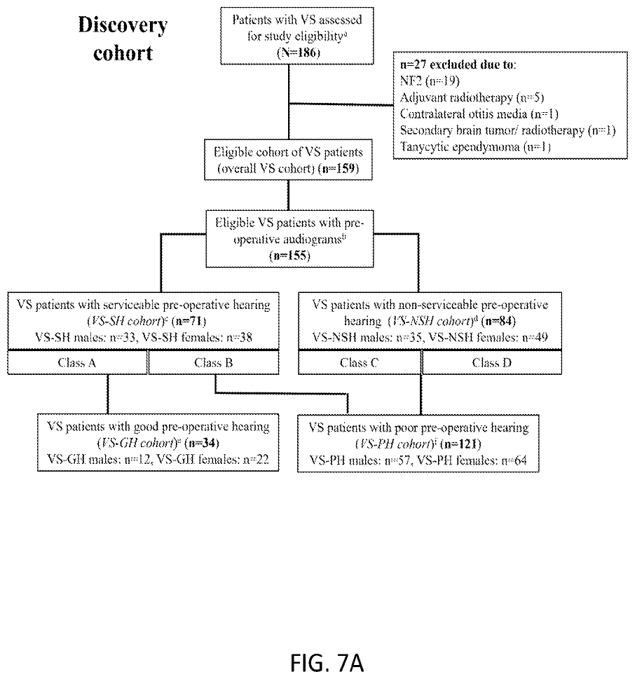

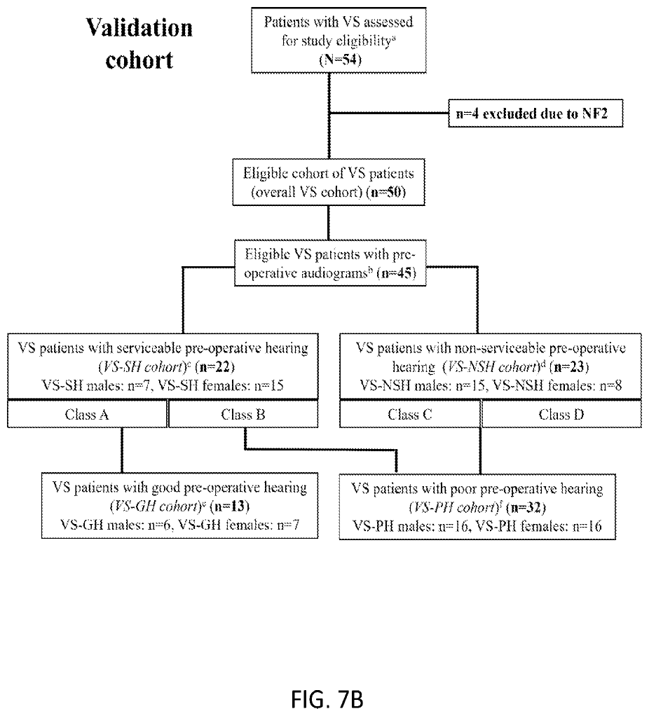

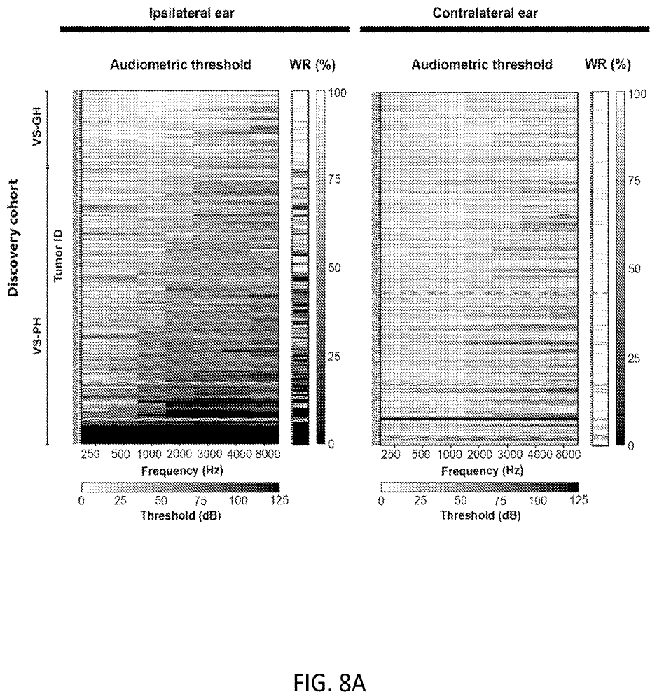

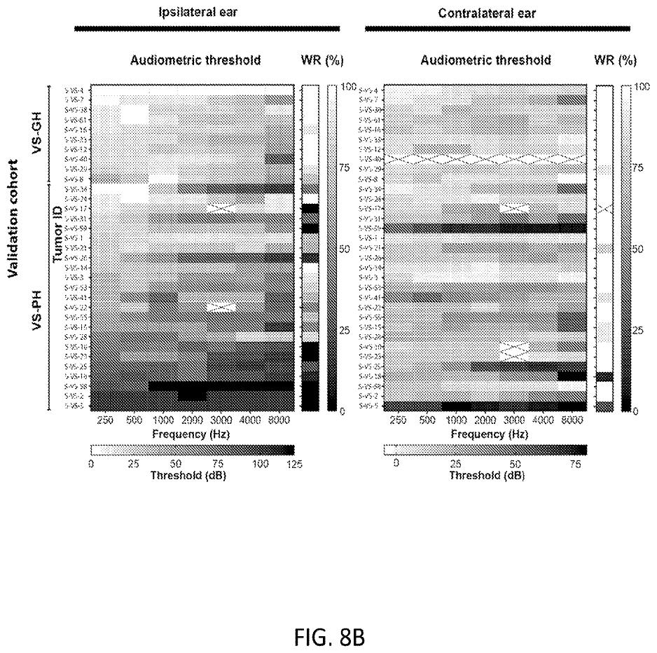

A total of 159 patients with sporadic VS, including 34 with GH and 121 with PH (FIG. 7A), were included for comparison with 70 controls. In this discovery cohort, VS patients and controls had similar mean ages (53 versus 46 years) and proportion of females (55 to 56%); VS-PH patients were significantly older than controls (53 years; P=0.002) (FIG. 1A). Compared with VS-GH patients, VS-PH patients had significantly larger tumor volume (6.9 versus 3.4 cm3, P=0.009), worse ipsilateral pure-tone average (PTA; 61.0 versus 17.3 dB) and word recognition percentage (WR; 39.6% versus 94.2%), and worse contralateral PTA (18.6 versus 8.6 dB; all P<0.005) (FIG. 8A).

In the validation cohort, a total of 50 patients with sporadic VS were studied, including 13 with GH and 32 with PH (FIG. 7B), and compared to 43 controls. VS patients and controls had similar mean ages (56 versus 53 years) but not proportion of females (52% versus 28%) (FIG. 1B). Compared with VS-GH patient, VS-PH patients had significantly worse ipsilateral PTA (60.1 versus 15.9 dB) and WR score (39.6% versus 94.9%; all P<0.005), and worse contralateral PTA (19.7 versus 10.1 dB; all P=0.015) (FIG. 8B). Worse hearing in contralateral ears of VS-PH patients is consistent with a previous report (21) and attributed to tumor-secreted ototoxic factors that may percolate through the cerebrospinal fluid of blood to reach the contralateral ear and affect its hearing.

### Plasma Levels of Candidate Biomarkers

### VS Patients Versus Controls

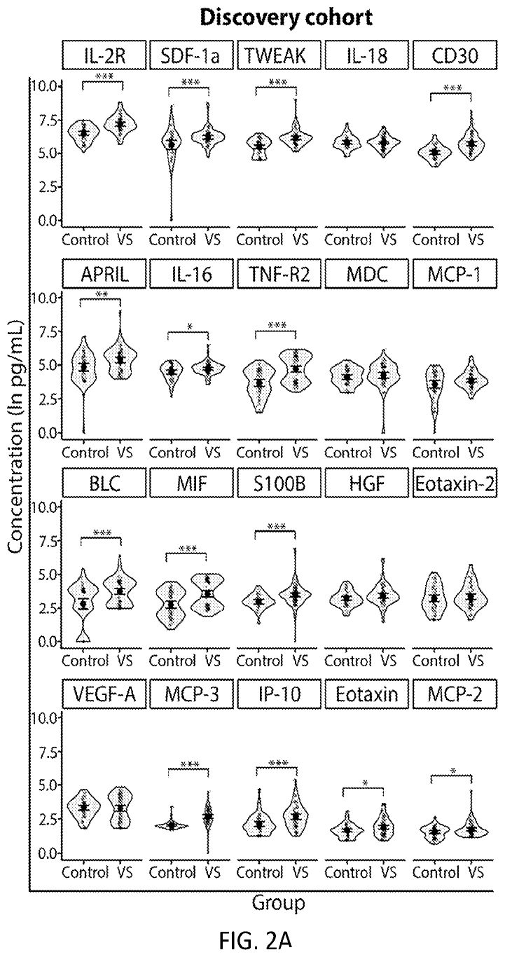

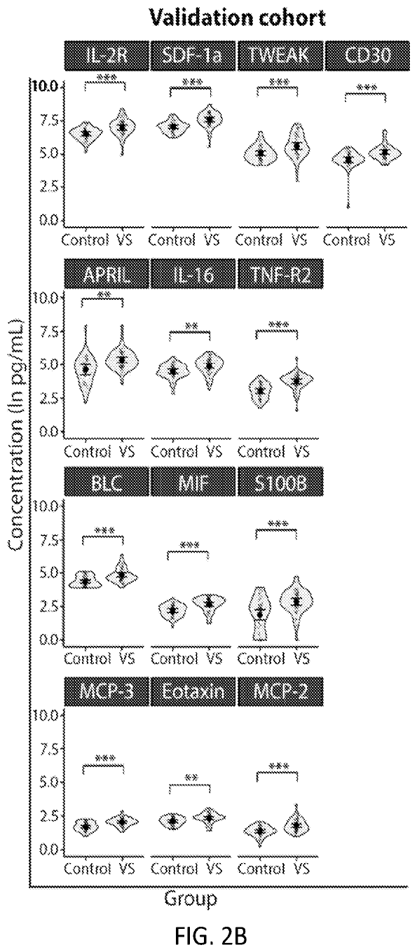

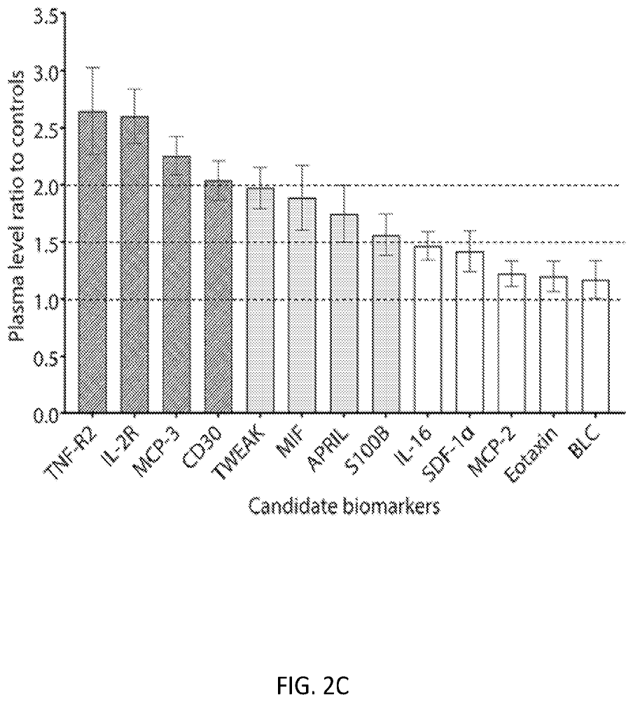

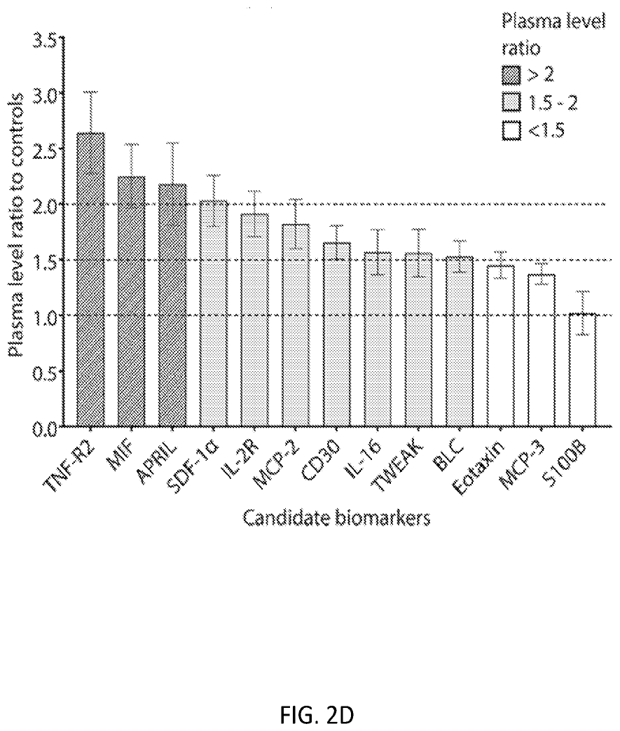

Twenty of the 66 profiled factors were detectable in the plasma of >75% of VS patients and considered candidate biomarkers (table S2). In both VS and control patients, the highest mean plasma values were for IL-2R, SDF-1a, and TWEAK, while the lowest values were for MCP-2, eotaxin, and MCP-3. The levels of candidate biomarkers measured in controls are in agreement with previous reports (22-26). The levels of 14 candidate biomarkers significantly differed between the VS and control groups in discovery cohort (FIG. 2A), and 13 of them were confirmed as significantly different in validation cohort (FIG. 2B). The most elevated factor among VS patients versus controls was TNF-R2 when adjusting for multiple comparisons (P_{adj}<0.001), where the ratio of plasma levels was ˜2.5 times higher in both cohorts (FIGS. 2C and 2D). TNF-α and FGF-2 were detected at very low levels in only 24% and 43% of analyzed patients, respectively, and were omitted from the analysis (table S2).

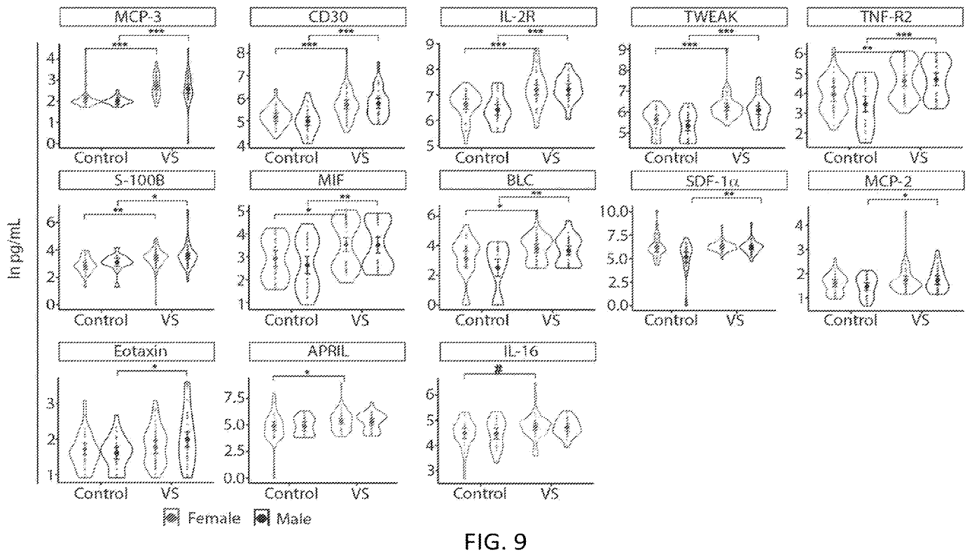

Eight validated biomarkers (MCP-3, CD30, IL-2R, TWEAK, TNF-R2, S100B, MIF, and BLC) were significantly elevated among VS patients of both sexes compared to controls (all P_{adj}<0.05) (FIG. 9). Eotaxin, MCP-2, and SDF-1a were significantly elevated only in male VS patients, while APRIL was only elevated in female VS patients (all P_{adj}<0.05).

### VS-GH and VS-PH Patients Versus Controls

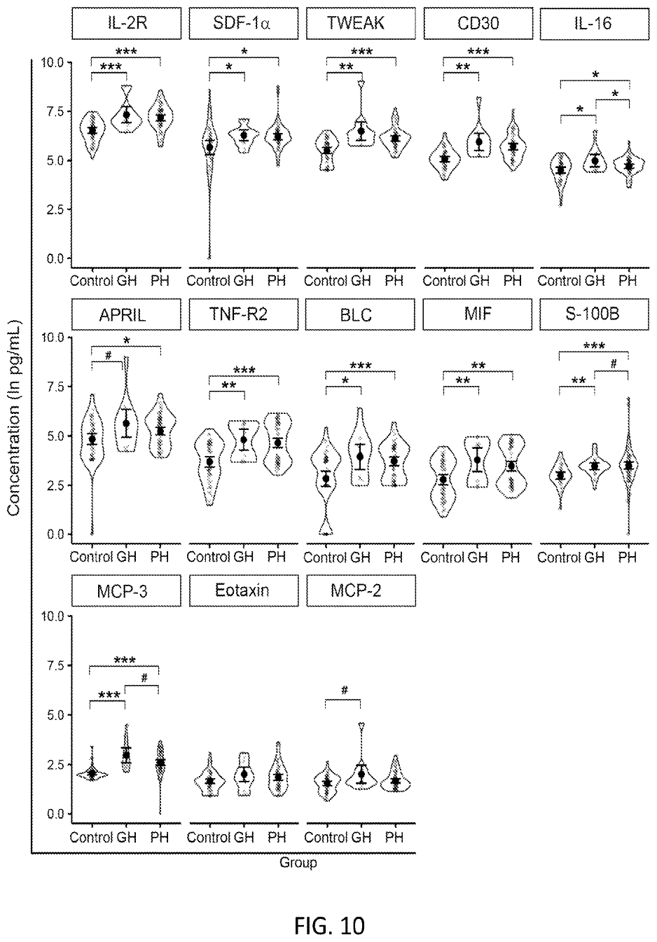

Compared to controls, 10 candidate biomarkers (MCP-3, CD30, S100B, TNF-R2, TWEAK, IL-2R, MIF, BLC, IL-16, and SDF-1a) were significantly elevated in both VS-GH and VS-PH patients, while APRIL and MCP-1 significantly differed only in VS-PH patients (FIG. 10). Controlling for sex and age, VS-GH patients had the highest increases of MCP-3 (45.10%) and BLC (39.50%).

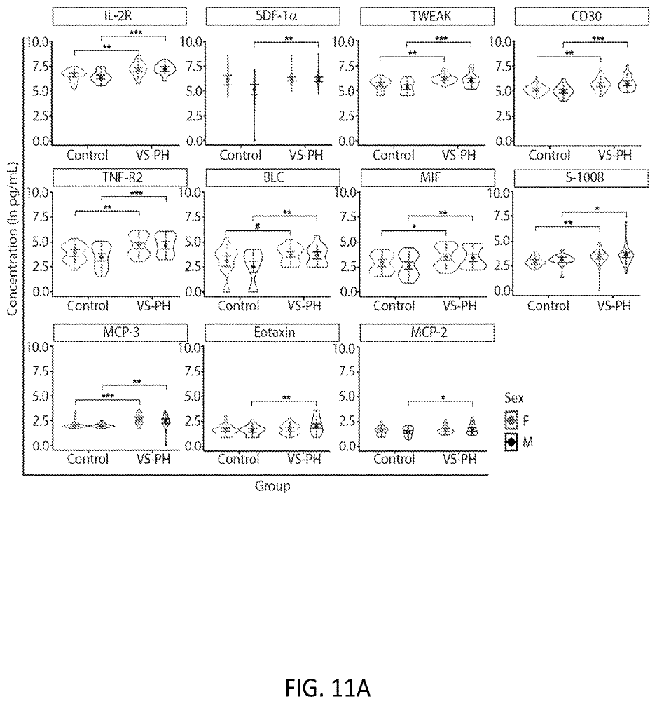

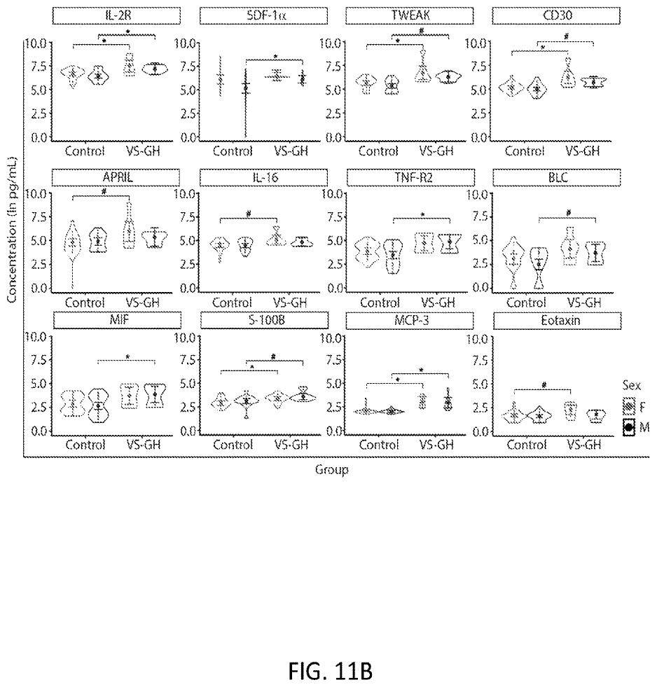

Compared to controls, there were significant sex simple effects among VS-PH patients of both sexes for levels of CD30, IL-2R, MCP-3, MIF, S100B, TNF-R2, and TWEAK, and among males only for BLC, eotaxin, MCP-1, MCP-2, and SDF-1a (P_{acj}<0.05) (FIG. 11A). There were significant sex simple effects among VS-GH patients of both sexes versus controls for IL-2R and MCP-3; among females only for CD30, S100B, and TWEAK; and among males only for MIF, SDF-1a, and TNF-R2 (all P_{adj}<0.05) (FIG. 11B).

### VS-GH Versus VS-PH Patients

The level of IL-16 was significantly higher in VS-GH versus VS-PH patients, and MCP-3 had the highest significant percent change between groups when controlling for sex, age, and tumor volume (both P_{adj}<0.05) (FIG. 10).

Interaction of Biomarker Levels with Preoperative Hearing

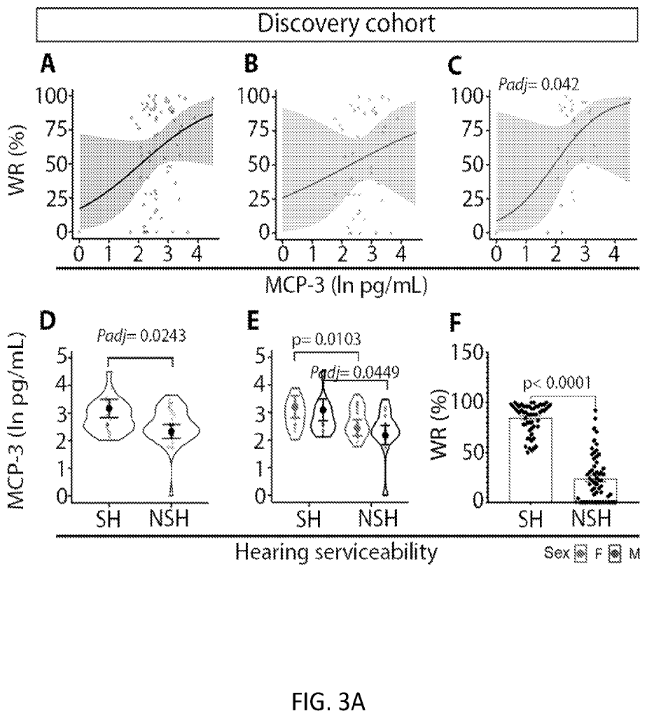

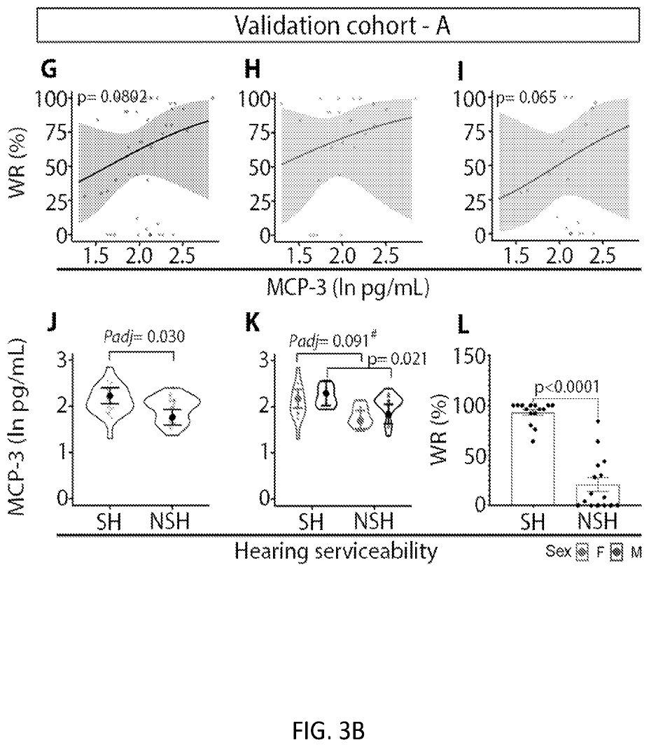

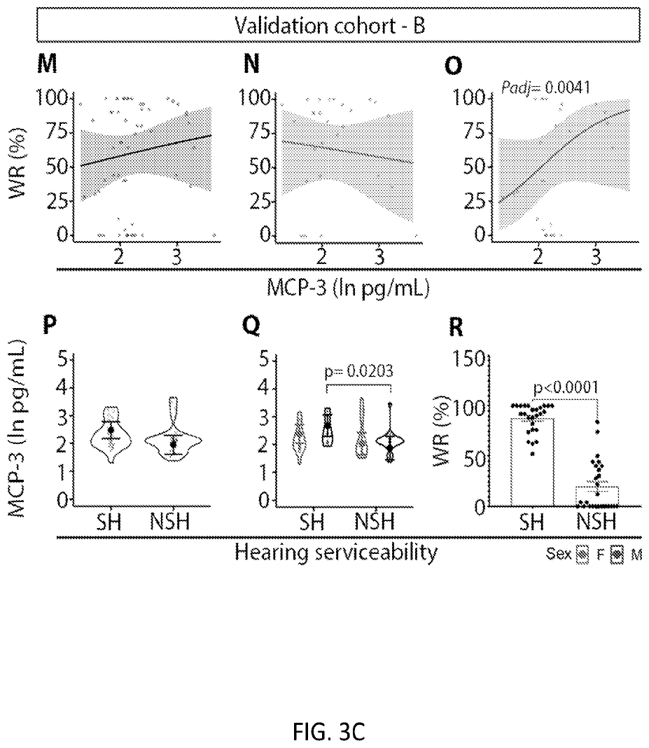

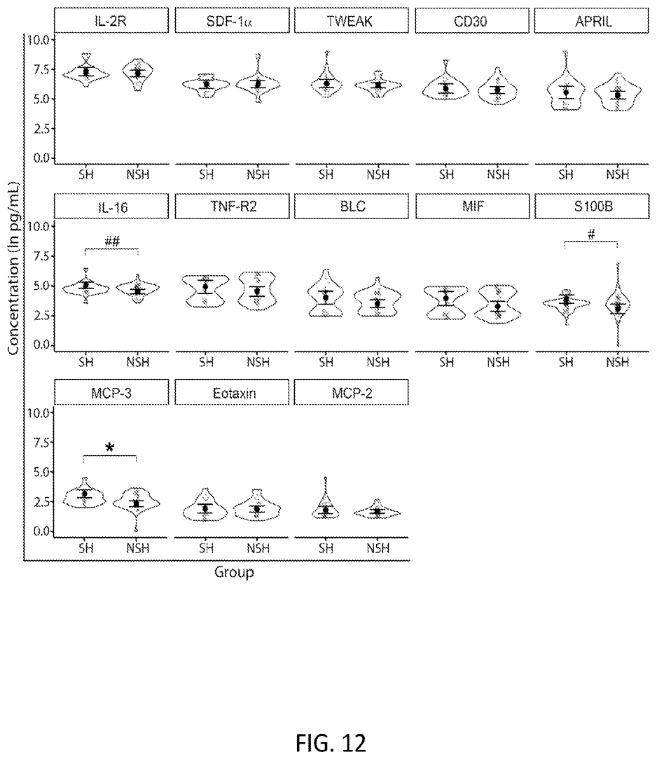

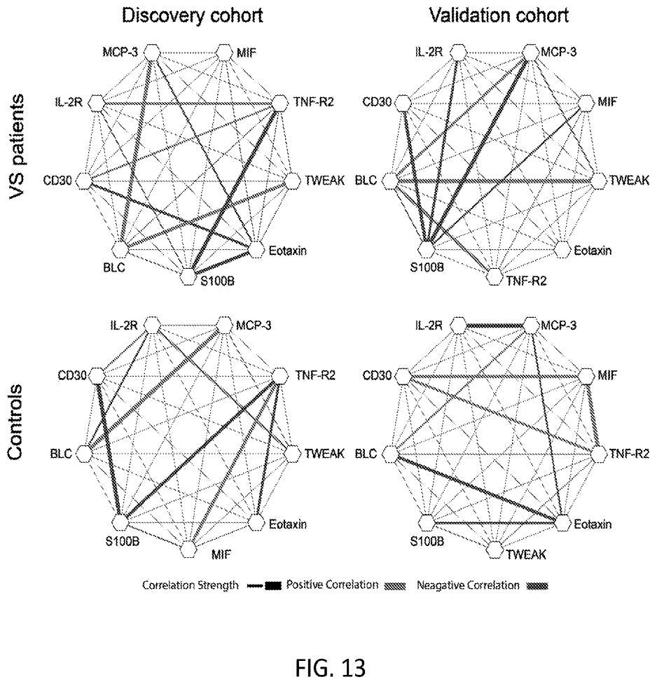

The relationships between candidate biomarker levels and preoperative PTA and WR were assessed among VS patients with a robust linear model (RLM) and a fractional logit model (FLM), respectively. There were no significant associations with PTA and biomarker levels, or sex-specific effects. However, there was a significant association between ipsilateral WR scores and MCP-3 levels (P_{adj}=0.042) in the discovery cohort of male VS patients (FIG. 3C relative to FIGS. 3A and 3B). According to the odds ratio, a 1-unit increase in MCP-3 natural log was associated with a WR increase of 135.5%. Furthermore, MCP-3 levels were significantly elevated in patients with serviceable hearing (SH) (P_{adj}=0.0243), defined as PTA≤50 dB and WR score ≥50% (FIG. 3D), and male VS patients (P_{adj}=0.0243) (FIG. 3E) whose WR scores were significantly higher compared to patients with non-SH (NSH) (FIG. 3F). In the validation cohort, the association between MCP-3 levels with WR scores showed the same trend as in the discovery cohort (FIGS. 3G to 31) and approached the criterion for significance (P=0.065 in male VS patients) (FIG. 31). The significantly elevated levels of MCP-3 in the patients with SH were confirmed in the validation cohort (FIGS. 3J and 3K). To test whether the trend in WR score and MCP-3 levels observed in the validation cohort (FIG. 31) would reach statistical significance by increasing sample size, the validation cohort (validation-A) was augmented by adding 13 randomly selected patients out of 78 VS patients from the discovery cohort to reach a balanced number of patients in both groups [65 and 63 patients in the discovery and balanced validation (validation-B) cohorts, respectively]. This analysis validated a significant association between WR score and MCP-3 levels in male VS patients (P_{adj}=0.0041) (FIG. 30).

Interaction of Biomarker Levels with Preoperative Tumor Volume

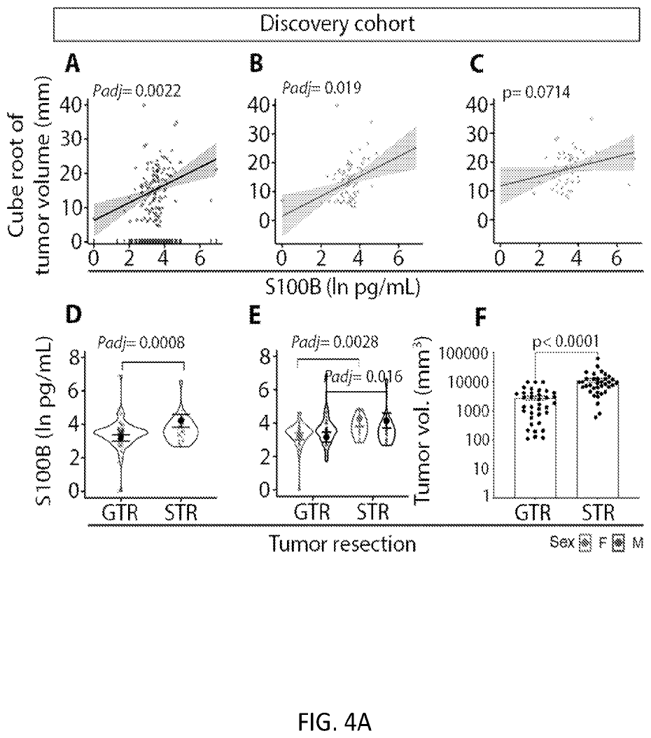

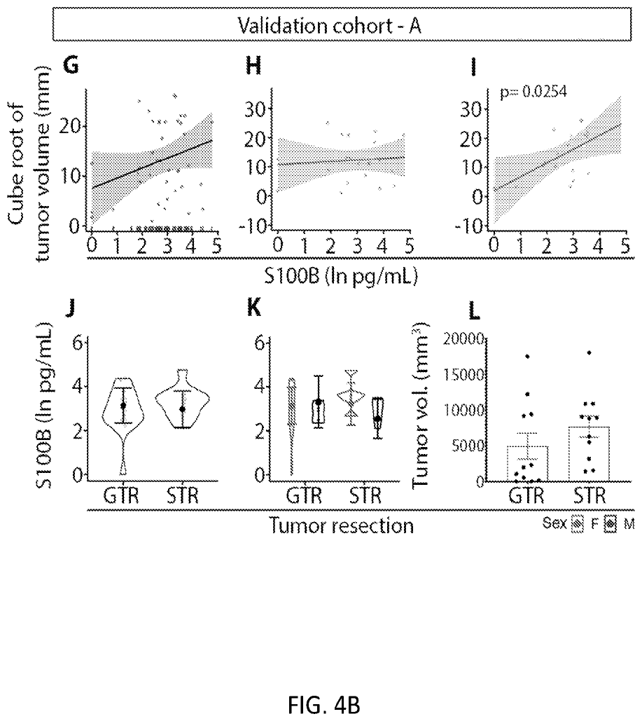

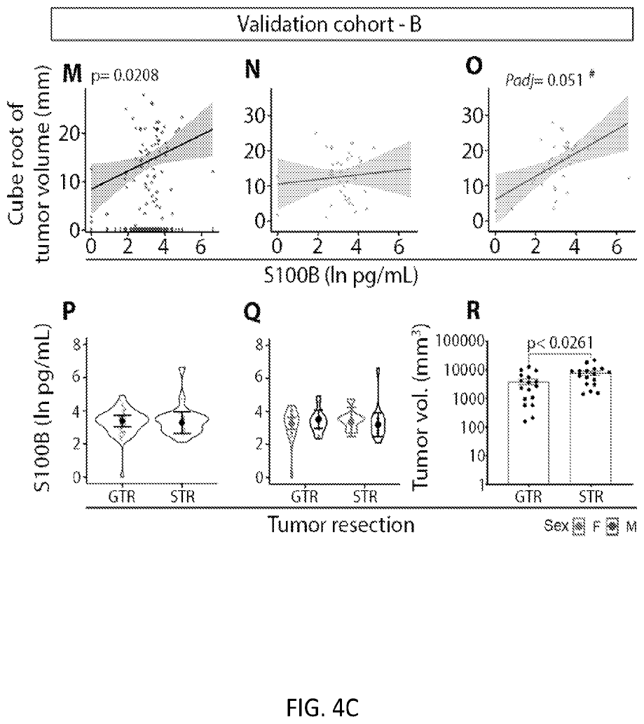

In the RLM assessing the relationship of biomarker levels and tumor volume, a significant association was found for S100B (FIG. 4A), with a significant sex effect among females (all P_{adj}<0.05) (FIG. 4B). Furthermore, S100B levels were significantly elevated in VS patients who underwent subtotal resection (STR) rather than gross total resection (GTR) of VS (FIGS. 4D and 4E). In addition, tumor volume was higher in VS patients who underwent STR compared to GTR (FIG. 4F). In the validation cohort-A, there was a trend for a positive association between S100B plasma levels and tumor volume in male VS patients (FIG. 41), which approached the criterion for statistical significance after correction for multiple comparisons by controlling the false discovery rate (FDR) to 0.10 (P=0.025, critical value: 0.023 in male VS patients). In the balanced validation cohort-B (increased to n=86 per the discovery cohort size), there was a significant association between S100B levels and tumor volume in male VS patients (P_{adj}=0.051 was considered significant for FDR of 0.10) (FIG. 40). However, the difference in S100B levels between the GTR and STR groups observed in the discovery cohort was not confirmed in the validation cohort, likely due to a smaller number of patients undergoing any VS surgical resection in the validation group (GTR, n=17; STR, n=11) (FIGS. 4J to 4L and 4P to 4R).

### Discriminative Power, Diagnostic Utility, and Relationships of Candidate Biomarkers

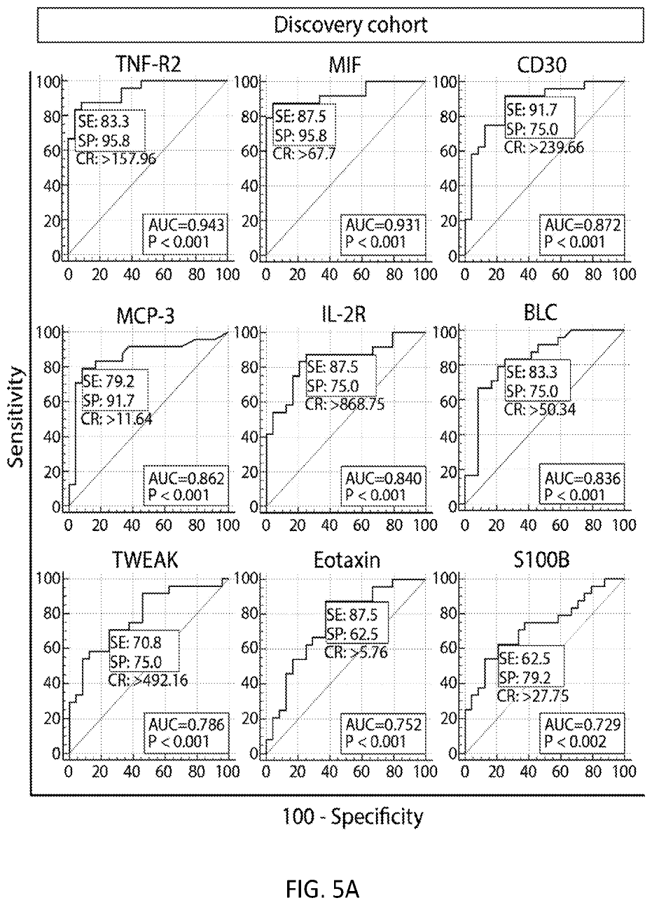

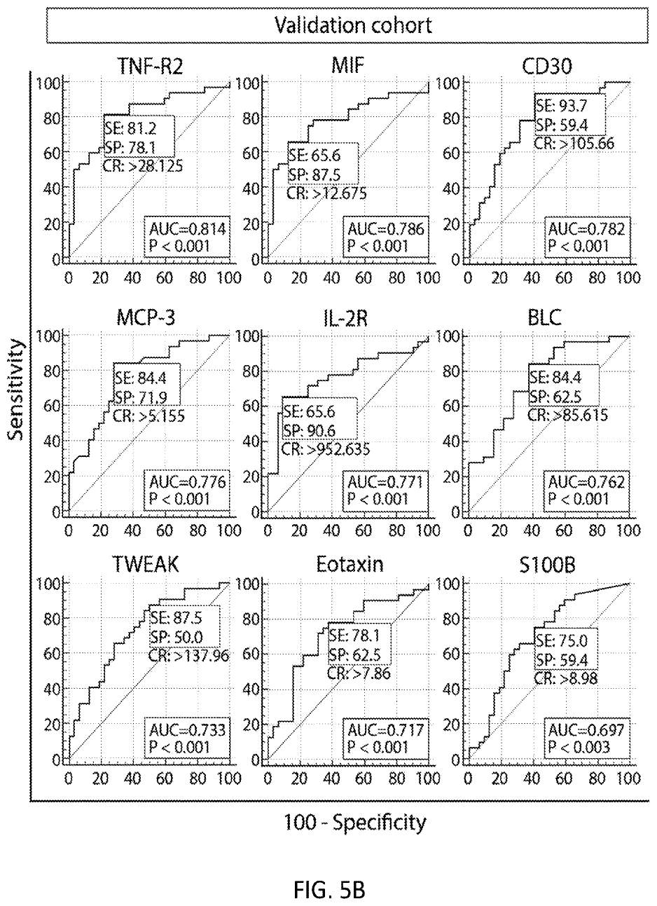

In the receiver-operating characteristic curve (ROC) analysis of the balanced dataset of sex- and age-matched VS patients and controls, in both the discovery and validation cohorts, 9 of the 13 significantly elevated factors in VS patients' plasma had a significant area under the curve (AUC) values. In the discovery cohort, AUC values were outstanding (0.9 to 1) for TNF-R2 and MIF; excellent (0.8 to 0.9) for CD30, MCP-3, IL-2R, and BLC; and acceptable (0.7 to 0.8) for TWEAK, eotaxin, and S100B. Although lower AUC values were observed in the validation cohort, the predictor capacity of tested biomarkers was still preserved, ranging from excellent for TNF-R2 to poor for S100B. In the validation cohort, TWEAK and eotaxin retained the unchanged predictor category, suggesting their predictor stability (FIG. 5). Moreover, the ranking of biomarkers in both cohorts remained unchanged, with TNF-R2 as a top biomarker and S100B with the lowest but statistically significant AUCs (0.729 and 0.697 in the discovery and validation cohorts, respectively). Consistent with our previously published data (9), the ROC analysis revealed that tumor size is not a robust biomarker of the hearing status in VS patients when comparing GH and PH (AUC=0.543), or SH and NSH (AUC=0.621).

To evaluate whether a biomarker panel could discriminate between VS patients and controls, a combinatorial analysis was performed using all nine validated biomarkers. The analysis revealed that combining nine biomarkers generated 502 panels ranging from two to nine biomarkers. Considering that MCP-3 and S100B are the only biomarkers significantly associated with patients' preoperative hearing and tumor size, 128 panels containing both MCP-3 and S100B were analyzed. The distribution of biomarkers was monitored in the top 15 panels given that they have the best performance (discovery cohort: AUC higher than 0.991, with 95.8 to 100% sensitivity and specificity; validation cohort: AUC higher than 0.872, with 78.1 to 93.8% sensitivity and 65.6 to 84.4% specificity). The most abundant biomarkers were TNF-R2 (discovery cohort) and IL-2R (both cohorts). They were followed by BLC, eotaxin, TWEAK, MIF, and CD30 in the discovery cohort, while IL-2R was followed by eotaxin, TWEAK, TNF-R2, BLC, and MIF in the validation cohort. In both cohorts, the nine-biomarker panel is among the top 15 panels. It shares the first place with 11 other panels in the discovery cohort (AUC=1, with 100% sensitivity and specificity) (table S3) and takes the sixth position in the validation cohort (AUC=0.874, with 90.6% sensitivity and 75% specificity) (table S4).

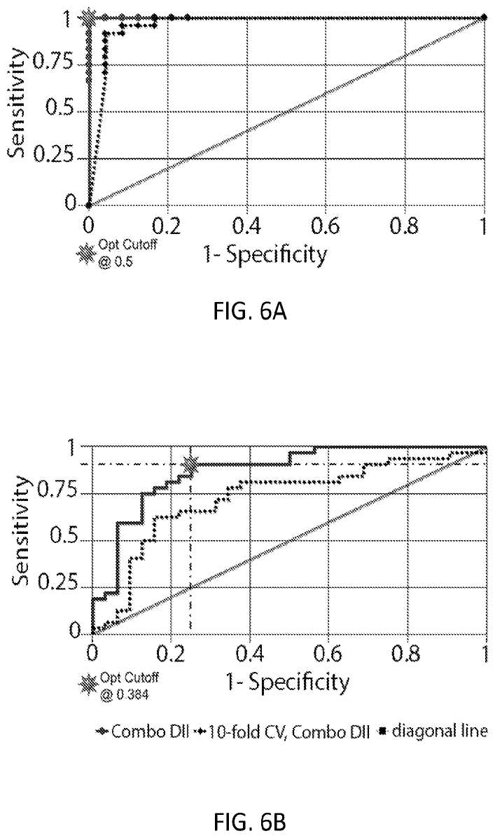

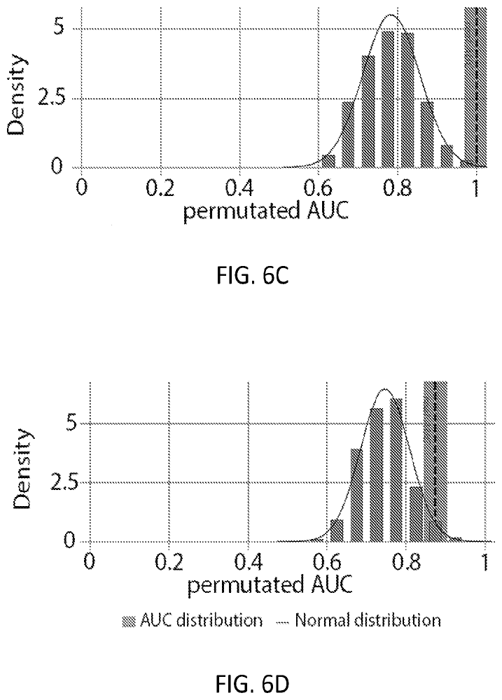

The nine-biomarker panel demonstrated significantly improved predictability compared to the mean AUC of the individual biomarkers (discovery cohort: AUC9-panel=1.000±0.000 versus AUCₘₑₐₙ of 9:0.839±0.025, P<0.0001; validation cohort: AUC9-panel=0.890±0.042 versus AUCₘₑₐₙ of 9:0.760±0.012, P=0.0029). This finding was confirmed by logistic regression analysis (table S5), and the accuracy and utility of the nine-biomarker panel were verified by performing 10-fold cross-validation and permutation tests (FIG. 6).

A mutual comparison of the nine candidate biomarkers with Spearman correlation analysis resulted in seven confirmed correlation pairs. Both VS patients and controls had significant positive correlations between (i) MCP-3 and BLC, (ii) CD30 and IL-2R, (iii) CD30 and MIF, and (iv) CD30 and TNF-R2. However, correlations between (i) BLC and TWEAK and (ii) CD30 and TWEAK were found mainly in VS patients, while the correlation between TNF-R2 and IL-2R was significant only in VS patients and not in controls, suggesting a disease-related association between these candidate biomarkers (FIG. S7). The strongest positive correlations in VS patients were between MCP-3 and BLC (discovery cohort, r=0.67; validation cohort, r=0.71) and BLC and TWEAK (discovery cohort, r=0.66: validation cohort, r=0.82) (all P<0.05). In healthy controls, the strongest positive correlation was observed between TNF-R2 and MIF (discovery cohort, r=0.61; validation cohort,r=0.87) (FIG. S7).

### Discussion

In this largest immune profiling to date of plasma from patients with sporadic VS, we identified 14 significantly elevated factors, 13 or which were verified in a separate cohort of VS patients. TNF-R2 was most highly elevated in VS patients, MCP-3 and S100B were associated with preoperative clinical characteristics, and nine factors showed a promising potential as circulating biomarkers.

Expression of TNF-R2 has been reported in many types of tumors, and serum concentration of soluble TNF-R2 is considered to be an indicator of TNF-R2 activity (27). Although TNF-R2 can be expressed on tumor cells, its main source may be highly suppressive regulatory T cells (Tregs) because the expansion of Tregs in cancer patients is followed by elevated serum values of soluble TNF-R2 (27), consistent with immune evasion. Given that we did not detect an association between TNF-R2 plasma levels and tumor size or hearing status in VS patients, elevated TNF-R2 may reflect an increased number of immunosuppressive cells in the VS patients' blood and more systemic immunosuppression than captured by tumor size or tumor-induced hearing loss. A significantly increased number of myeloid-derived suppressor cells have been reported in the blood of VS patients with NF2-related schwannomatosis (28). Future studies should determine whether there is a correlation between plasma levels of TNF-R2 and the number of circulatory Tregs in VS patients. In the meantime, our data support the notion of systemic immunosuppression in patients with sporadic VS because we found a significant positive correlation between TNF-R2 and IL-2R (r=0.591, P=0.002), known to be abundantly expressed on Tregs (29). Relevantly, intracranial malignancies have been previously associated with systemic immunosuppression (30), which in turn has been associated with hearing impairment (31).

MCP-3 is an important immune-related factor whose relationship with GH is highlighted by our study. We showed that, in VS patients, MCP-3 is significantly elevated among those with SH and that higher MCP-3 levels were associated with better WR scores. It is currently unknown whether MCP-3, a proinflammatory chemokine with a prominent role in tumorigenesis (32), could be involved in hearing protection. Considering that this chemokine performs its function by binding to four receptors (CCR1, CCR2, CCR3, and CCR5), which are shared with other chemokines (33), MCP-3 may antagonize other chemokines with potential ototoxic activity. It is interesting that a receptor for MCP-3 is CCR2, which is reported to play a protective role against noise-induced cochlear hair cell death (34). Moreover, DA RC, an atypical chemokine receptor that can bind MCP-3, is broadly expressed in the mouse cochlea, including hair cells, supporting cells, and spiral ganglion neurons (35). This finding suggests that tumor-secreted MCP-3 may affect cochlear function.

S100B is a candidate biomarker whose plasma values were significantly associated with tumor volume. S100B is broadly expressed in Schwann cells whose proliferation, myelinating activity, and S100B expression are controlled by the SOX-10 transcription factor (36). Reduced expression of SOX-10 at the protein and gene levels has been demonstrated in human schwannoma cells, while the loss of SOX-10 expression in Schwann cells leads to them acquiring schwannoma cell characteristics (37). While the direct effect of S100B on schwannoma growth has not been studied mechanistically, it has been established that the protumorigenic effect of S100B in melanoma and glioma involves inhibition of p53 tumor suppressor protein activity, stimulation of mitogenic kinases activity (Ndr and Akt), and macrophage chemoattraction (38, 39). A correlation between S100B serum levels and tumor growth has been reported in melanoma patients, where decreased levels were associated with clinical response to therapy (40). For VS, there are two reports showing opposite outcomes. Similar to us, Kanner et al. (41) reported a significant positive correlation between serum S100B levels and tumor size only for VS tumors (n=6) but not for all other analyzed solid tumors, including glial tumors (n=8), metastatic tumors (n=27, predominantly lung and breast carcinoma), meningiomas (n=8), and chondroma (n=1). However, Smith et al. (14) reported that plasma S100B was not a useful biomarker for tumor burden in neurofibromatosis patients (NF1-related schwannomatosis: n=69; NF2-related schwannomatosis: n=28; schwannomatosis: n=30) because they did not find a relationship between the presence of internal neurofibromas or schwannomas and S100B plasma levels, or between whole-body tumor burden and S100B concentration. However, Smith et al. noted the limited ability of whole-body MRI to image the internal auditory canal, which prevented them from confirming any relationship between VS size and S100B levels in patients with NF2-related schwannomatosis (14). Together, our plasma findings measured in 122 patents with sporadic VS, along with the results of Kanner et al., suggest the utility of S100B plasma levels as a biomarker associated with tumor size in VS patients.

Finally, we demonstrated and validated the diagnostic utility of 9 of 13 immune-related factors with confirmed significantly elevated levels in VS patients. We proposed a nine-biomarker panel and showed its excellent discrimination ability for VS. TNF-R2 was chosen as the highly elevated biomarker in VS patients, MCP-3 was selected as the only verified factor significantly associated with SH and WR, S100B is a phenotypic marker of VS correlating with tumor size, and MIF, CD30, IL-2R, BLC, TWEAK, and eotaxin were selected based on their contribution to improving the predictability (panel AUC) of the other biomarkers. Correlation analysis showed significant interconnections between the panel biomarkers, suggesting possible roles in VS pathogenesis. Overall, our findings suggest that the nine-biomarker panel could be an additional tool to monitor or predict hearing change and tumor growth in VS patients and help inform diagnoses or the ideal timing of tumor resection to preserve hearing.

One limitation of our study is that it is cross-sectional, and therefore, the temporal link between the outcome and VS presence cannot be determined because both are examined simultaneously. A future longitudinal study focused on the validation of our findings is suggested. Such a study may benefit from multi-institutional patient accrual because VS is a rare tumor. Another limitation of our study is the lack of quantitative hearing assessment in control patients because their deidentified blood was collected from blood donor centers. However, we quantified hearing in ears contralateral to VS (FIG. 7), and hearing in these non-tumor-bearing ears of VS-GH patients should be representative of the control population that is similar in age and sex distributions, as we previously showed (21). In that study, we also showed that VS-PH patients have long-term risk of progression to moderate hearing loss in the contralateral ear as well, which typically manifests 12 years after VS-PH diagnosis (21). We validated differences in candidate biomarkers between VS patients and controls in two different patient populations and two different laboratories. Nonetheless, a future study involving quantitative audiometric data for all control subjects is suggested to discern how hearing status may influence immune-related molecules in plasma.

In summary, this study describes the robust immune profiling of blood plasma from a large cohort of patients with sporadic VS for comparison with controls, between VS-GH and VS-PH patients, between VS patients with SH and NSH, and between sexes. We correlated biomarker levels with presurgical PTA, WR, and tumor size and validated a potentially diagnostic nine-biomarker composite panel with outstanding/excellent discriminatory ability for VS.

### Materials and Methods

### Study Population and Specimen Collection

From July 2015 to April 2021, blood was prospectively collected from patients undergoing VS resection at Massachusetts Eye and Ear (ME E) in Boston, MA on the day of surgery, typically within 30 min of inducing general anesthesia and ≥1 hour before tumor microdissection (discovery cohort). Blood was similarly prospectively collected from August 2021 to June 2023 at Stanford Hospital in Palo Alto, CA (validation cohort). Blood from controls was collected at the Massachusetts General Hospital (MGH) Blood Donor Center in Boston, MA, and the Research Blood Components in Watertown, MA for the discovery cohort, and from the Stanford Hospital Blood Donor Center in Palo Alto, CA for the validation cohort. At collection, fresh blood was stored in E DTA vacutainer tubes (Becton Dickinson, NY, USA) and kept at 4° C. without freezing. The whole blood samples were centrifuged at 2000 g for 10 min at 4° C. Plasma was separated and spun at 2000 g for 5 min at 4° C. Centrifuged plasma was filtered through 0.8-μm filter units (MF-Millipore MCE membrane, SLA A033SB; Millipore, Burlington, MA, USA) and stored at −80° C. until further use.

Eligible patients had unilateral, sporadic VS that had not been previously resected or irradiated. Of 186 and 50 enrolled VS patients in the discovery and validation cohorts, 163 and 50 met inclusion criteria, respectively, and were included in the analyses (CONSORT diagram in FIG. 7) for comparison with 70 and 43 controls, respectively.

### Clinical Data

Clinical and demographic data were collected from patient charts, operative reports, pathology reports, and preoperative radiographic imaging. Patient variables included age at tissue collection, presurgical tumor volume measured via high-resolution axial contrast-enhanced T1-weighted brain MRI, internal auditory canal protocol (42), and presurgical pure-tone audiometric threshold and WR measurements. MRI and hearing tests were those nearest to resection, typically ≤3 months. WR was defined as the percentage of spoken monosyllabic words discernable from a list typically read at 70 dB or the level at which a patient's speech intelligibility curve plateaus. Pure-tone audiometric thresholds at 0.5, 1, 2, and 3 kHz were used to calculate the PTA. Hearing groups were defined according to the American Academy of Otolaryngology-Head and Neck Surgery (AAO-HNS) Hearing Classification Guidelines (43). GH was defined as WR >70% and PTA <30 dB (AAO-HNS class A hearing). Otherwise, patients were classified as having PH (AAO-HNS class B, C, and D hearing). A deaf ear was assigned a PTA of 125 dB and WR score of 0%. SH was defined as WR ≥50% and PTA≤50 dB (AAO-HNS class A and B hearing). Otherwise, patients were classified as having NSH (AAO-HNS class C and D hearing). Tumor resection was defined as GTR if there was complete tumor removal or a small tumor remnant no greater than 5×5×2 mm was left behind to preserve nerve integrity (17). Otherwise, it was defined as STR.

### Biomarker Measurements

Luminex (65 cytokines, chemokines, growth factors, and soluble receptors), electrochemiluminescence (IL-18, TNF-α, and FGF-2), and ELISA (enzyme-linked immunosorbent assay) (S100B) assays were conducted as described in detail below. Potential biomarkers were defined as those detectable in the plasma of >75% of patients. The list of analyzed biomarkers is in table S1.

### Luminex Assay

Simultaneous multiplex profiling of 65 immune-related factors composed of cytokines, chemokines, and growth factors was performed using a customized multiplex bead-based immunoassay—Immune Monitoring 65-Plex Human ProcartaPlex Panel (#E PX650-10065-901; Thermo Fisher Scientific, Waltham, MA, USA) according to the manufacturer's instructions. The fluorescence-based signal was acquired on the Magpix instrument (Luminex, Austin, TX, USA), and the values of analytes were calculated using ProcartaPlex Analyst 1.0 Software (Thermo Fisher Scientific). The analytes with values below the lower limit of quantification in more than 75% of all samples were excluded from the analysis (table S1). For the accepted analytes, the values between 0 μg/ml and the lower limit of quantification, and those exceeding the upper limit of quantification, were approximated with the lowest and highest concentration representing these limits, respectively.

### Electrochemiluminescence Assay

The following candidate biomarkers were measured using electrochemiluminescence-based human assays from Meso Scale Diagnostics (MSD; Rockville. MD, USA): IL-18 (U-PLEX Human IL-18) and TNF-α (U-PLEX Human TNF-α). All assays were conducted according to the manufacturer's protocols, and signal detection was performed on the QuickPlex SQ 120 device (MSD). Preanalytical data processing was done using MSD Discovery Workbench software (v4.0.12).

### Enzyme-Linked Immunosorbent Assay

The S100B ELISA Kit (#E Z H S100B-33K; E MD Millipore, Billerica, MA, USA) was used to measure S100B protein levels in the plasma of VS patients and controls. ELISAs were performed by adhering to the manufacturer's protocol. Absorbance was measured using SpectraMax 190 plate Reader (Molecular Devices, Sunnyvale, CA, USA), and the standard curve was plotted using SoftMax Pro software (v5.2; Molecular Devices, San Jose, CA, USA).

### Statistics

Patient group comparisons comprised (i) all VS patients versus controls), (ii) VS-GH patients versus controls, (iii) VS-PH patients versus controls, (iv) VS-PH versus VS-GH patients, (v) VS-SH versus VS-NSH, and (vi) VS patients undergoing GTR versus STR. For all comparisons, P<0.05 was considered statistically significant. P values were corrected for multiple comparisons (and called P_{adj}) using the Benjamini-Hochberg FDR procedure. Differences in age across groups were analyzed using one-way analysis of variance (A NOVA) with a Dunn's multiple comparisons test. Differences in tumor volume and PTA were analyzed using Mann-Whitneyt test. Differences in WR were analyzed using N-1 chi-square test for the comparison of two proportions expressed as a percentage. The ratio method was used to assess the individual effect of candidate biomarkers on the WR of VS patients.

### Generalized Linear Mixed-Effects Regression

Generalized linear mixed-effects regression models were used to assess the relationship between candidate biomarkers' levels in the plasma of VS patients and controls versus clinical variables. All candidate biomarkers were natural log transformed before modeling. To compare plasma biomarker levels in VS patients with controls while controlling for subjects' age and sex, the following generalized least squares (GLS) model was used: In <biomarker>=βb0+βb1 Sex+βb2Age+bβ3GH+βb4PH. The biomarker in the formula refers to tested immune-related factors, while βb0, βb1, βb2, βb3, and βb4 are regression coefficients representing the change in natural log-transformed concentrations of prospective biomarkers to a one-unit change in the respective independent variable (sex, age, GH, and PH). The effect of sex x candidate biomarker interactions was assessed by the same model with the following terms: In <biomarker>=βb0+βb1 Sex+βb2Age+βb3GH+βb4PH+βb5Sex x* In <biomarker>. For mutual comparison of GH and PH groups, the GLS model was extended for controlling the subject's tumor volume.

RLMs were used to regress ipsilateral PTA values on plasma biomarker levels while controlling for sex, age, tumor volume, and PTA contralateral to VS. RLMs were also used to regress tumor volume on plasma biomarker levels while controlling for sex and age. The use of RLMs was necessary for both outcomes, as non-RLMs suffered from substantial effect size biases caused by residual outliers. RLM was used to control for patients' age, sex, tumor size, and hearing in the contralateral ear. The model had the following terms: PTA_ipsi=βb0+βb1Sex+βb2Age+bβ3PTA_contra+βb4 Tumor volume\hat{ }(1/3)+βb5In <biomarker>, where PTA_ipsi is PTA in the ear ipsilateral to the tumor and PTA_contra is PTA in the ear contralateral to the tumor. To investigate the effect of sex x candidate biomarker interactions, the same RLM was used with the following terms: PTA_ipsi=βb0+βb1Sex+βb2Age+βb3PTA_contra+βb4 Tumor volume\hat{ }(1/3)+βb5/n<biomarker>+βb6Sex x* In <biomarker>. The regression coefficients (βb0, βb1, b2, b3, βb4, βb5, and βb6) represent the change in PTA_ipsi to a one-unit change in the respective independent variable (sex, age, PTA_contra, tumor volume, biomarker, and interactions of sex and biomarker concentration).

In the analysis of the interaction with tumor volume, the RLM had the following terms. Tumor volume\hat{ }(1/3)=βb0+βb1 Sex+βb2Age+βb3In <biomarker>, where βb0, βb1, βb2, and βb3 are regression coefficients representing the change in tumor volume to a one-unit change in the respective independent variable (sex, age, and biomarker concentration). To investigate the effect of sex x candidate biomarker interactions, the same RLM was extended as follows: Tumor volume\hat{ }(1/3)=b30+βb1 Sex+βb2Age+βb3In <biomarker>+βb4Sex x*<In biomarker>, where bβ4 is a regression coefficient representing the change in tumor volume to a one-unit change in the variable determined by interactions of sex and biomarker concentration.

FLMs were used to regress WR scores ipsilateral to VS on plasma biomarker levels while controlling for sex, age, tumor volume, and WR scores contralateral to VS. Fractional models were necessary since WR scores were measured on the interval [0, 100] and therefore had hard upper and lower bounds, as well as being heteroskedastic. FLM was used to control for patients' age, sex, tumor size, and hearing in the contralateral ear. The model had the following terms: WR_ipsi=βb0+βb1 Sex+βb2Age+βb3WR_contra+βb4Tumor volume\hat{ }(1/3)+βb5/n<biomarker>, where WR_ipsi is WR in the ear ipsilateral to the tumor and WR_contra is WR in the ear contralateral to the tumor. The effect of sex x candidate biomarker interactions was assessed by the same model with the following terms: WR_ipsi=βb0+βb1 Sex+βb2Age+βb3 WR_contra+βb4 Tumor volume\hat{ }(1/3)+βb5/n<biomarker>+βb6 Sex x* In <biomarker>. The regression coefficients (βb0, βb1, βb2, βb3, βb4, βb5, and βb6) represent the change in WR_ipsi to a one unit change in the respective independent variable (sex, age, WR_contra, tumor volume, biomarker, and interactions of sex and biomarker concentration).

ROC analysis was used to evaluate the diagnostic power and utility of significantly elevated candidate plasma biomarkers. The ROC analysis was performed on the balanced dataset of sex- and age-matched VS patients and controls, and the areas under the ROC curve (AUCs) were calculated using MedCalc software. The discriminatory power of candidate biomarkers was categorized as follows: “outstanding” discrimination, AUC≥0.90; “excellent” discrimination, 0.80≤AUC<0.90; “acceptable” discrimination, 0.70≤AUC<0.80; and “poor” discrimination, AUC<0.70 (44).

### Statistical Software

Comparisons of demographics, tumor volume, and hearing loss between groups were performed with GraphPad Prism (v9.3.1; GraphPad Software, La Jolla, CA, USA) and MedCalc Statistical Software (v20.109; MedCalc Software Ltd., Ostend, Belgium). Models were estimated, and graphs were generated using R (v3.6.3; R Foundation, Vienna, Austria). Combinatorial analysis of candidate biomarkers was performed using the CombiROC web application (combiroc.eu) (45) and was assessed by Spearman correlation analysis using GraphPad software.

### Study Approval

All study protocols were approved by the Human Studies Committee of MEEI and MGH [Institutional Review Board (IRB) protocol #14-148H] as well as Stanford (IRB protocol #60363). All participants provided written informed consent before participation.

### REFERENCES

1 M. S. Mahaley Jr., C. Mettlin, N. Natarajan, E. R. Laws Jr., B. B. Peace, Analysis of patterns of care of brain tumor patients in the United States: A study of the Brain Tumor Section of the AANS and the CNS and the Commission on Cancer of the ACS. Clin. Neurosurg. 36, 347-352 (1990).
2 R. A. Sobel, Vestibular (acoustic) schwannomas: Histologic features in neurofibromatosis 2 and in unilateral cases. J. Neuropathol. Exp. Neurol. 52, 106-113 (1993).
3 J. L. Fisher, D. Pettersson, S. Palmisano, J. A. Schwartzbaum, C. G. Edwards, T. Mathiesen, M. Prochazka, T. Bergenheim, R. Florentzson, H. Harder, G. Nyberg, P. Siesjö, M. Feychting, Loud noise exposure and acoustic neuroma. Am. J. Epidemiol. 180, 58-67 (2014).
4 S. R. Plotkin, L. Messiaen, E. Legius, P. Pancza, R. A. Avery, J. O. Blakeley, D. Babovic-Vuksanovic, R. Ferner, M. J. Fisher, J. M. Friedman, M. Giovannini, D. H. Gutmann, C. O. Hanemann, M. Kalamarides, H. Kehrer-Sawatzki, B. R. Korf, V. F. Mautner, M. MacCollin, L. Papi, K. A. Rauen, V. Riccardi, E. Schorry, M. J. Smith, A. Stemmer-Rachamimov, D. A. Stevenson, N. J. Ullrich, D. Viskochil, K. Wimmer, K. Yohay; International Consensus Group on Neurofibromatosis Diagnostic Criteria (I-NF-DC), S. M. Huson, P. Wolkenstein, D. G. Evans, Updated diagnostic criteria and nomenclature for neurofibromatosis type 2 and schwannomatosis: An international consensus recommendation. Genet. Med. 24, 1967-1977 (2022).
5 C. Matthies, M. Samii, Management of 1000 vestibular schwannomas (acoustic neuromas): Clinical presentation. Neurosurgery 40, 1-9 (1997).
6 D. J. Genther, K. D. Frick, D. Chen, J. Betz, F. R. Lin, Association of hearing loss with hospitalization and burden of disease in older adults. JAMA 309, 2322-2324 (2013).
7 A. Mohammadi, N. Jufas, Sudden death due to vestibular schwannoma: Caution in emergent management. Otol. Neurotol. 37, 564-567 (2016).
8 E. E. Smouha, M. Yoo, K. Mohr, R. P. Davis, Conservative management of acoustic neuroma: A meta-analysis and proposed treatment algorithm. Laryngoscope 115, 450-454 (2005).
9 A. Brown, S. Early, S. Vasilijic, K. M. Stankovic, Sporadic vestibular schwannoma size and location do not correlate with the severity of hearing loss at initial presentation. Front. Oncol. 12, 836504 (2022).
10 B. Akpinar, S. H. Mousavi, M. M. McDowell, A. Niranjan, A. H. Faraji, J. C. Flickinger, L. D. Lunsford, Early radiosurgery improves hearing preservation in vestibular schwannoma patients with normal hearing at the time of diagnosis. Int. J. Radiat. Oncol. Biol. Phys. 95, 729-734 (2016).
11 M. E. Sughrue, A. J. Kane, R. Kaur, J. J. Barry, M. J. Rutkowski, L. H. Pitts, S. W. Cheung, A. T. Parsa, A prospective study of hearing preservation in untreated vestibular schwannomas. J. Neurosurg. 114, 381-385 (2011).
12 M. E. Sughrue, I. Yang, D. Aranda, K. Lobo, L. H. Pitts, S. W. Cheung, A. T. Parsa, The natural history of untreated sporadic vestibular schwannomas: A comprehensive review of hearing outcomes. J. Neurosurg. 112, 163-167 (2010).
13 S. R. Plotkin, J. O. Blakeley, E. Dombi, M. J. Fisher, C. O. Hanemann, K. S. Walsh, P. L. Wolters, B. C. Widemann, Achieving consensus for clinical trials: The REINS International Collaboration. Neurology 81( ) S1-S5 (2013).
14 M. J. Smith, S. Esparza, V. L. Merker, A. Muzikansky, M. A. Bredella, G. J. Harris, A. Kassarjian, W. Cai, J. A. Walker, V. F. Mautner, S. R. Plotkin, Plasma S100β is not a useful biomarker for tumor burden in neurofibromatosis. Clin. Biochem. 46, 698-700 (2013).
15 J. O. Blakeley, X. Ye, D. G. Duda, C. F. Halpin, A. L. Bergner, A. Muzikansky, V. L. Merker, E. R. Gerstner, L. M. Fayad, S. Ahlawat, M. A. Jacobs, R. K. Jain, C. Zalewski, E. Dombi, B. C. Widemann, S. R. Plotkin, Efficacy and biomarker study of bevacizumab for hearing loss resulting from neurofibromatosis type 2-associated vestibular schwannomas. J. Clin. Oncol. 34, 1669-1675 (2016).
16 S. R. Plotkin, D. G. Duda, A. Muzikansky, J. Allen, J. Blakeley, T. Rosser, J. L. Campian, D. W. Clapp, M. J. Fisher, J. Tonsgard, N. Ullrich, C. Thomas, G. Cutter, B. Korf, R. Packer, M. A. Karajannis, Multicenter, prospective, phase II and biomarker study of high-dose bevacizumab as induction therapy in patients with neurofibromatosis type 2 and progressive vestibular schwannoma. J. Clin. Oncol. 37, 3446-3454 (2019).
17 Y. Ren, H. Hyakusoku, J. E. Sagers, L. D. Landegger, D. B. Welling, K. M. Stankovic, MMP-14 (MT1-MMP) is a biomarker of surgical outcome and a potential mediator of hearing loss in patients with vestibular schwannomas. Front. Cell. Neurosci. 14, 191 (2020).
18 S. Dilwali, A. Lysaght, D. Roberts, F. G. Barker II, M. J. McKenna, K. M. Stankovic, Sporadic vestibular schwannomas associated with good hearing secrete higher levels of fibroblast growth factor 2 than those associated with poor hearing irrespective of tumor size. Otol. Neurotol. 34, 748-754 (2013).
19 S. Dilwali, L. D. Landegger, V. Y. Soares, D. G. Deschler, K. M. Stankovic, Secreted factors from human vestibular schwannomas can cause cochlear damage. Sci. Rep. 5, 18599 (2015).
20 J. E. Sagers, M. I. Sahin, I. Moon, S. G. Ahmed, A. Stemmer-Rachamimov, G. J. Brenner, K. M. Stankovic, NLRP3 inflammasome activation in human vestibular schwannoma: Implications for tumor-induced hearing loss. Hear. Res. 381, 107770 (2019).
21 S. Early, C. E. Rinnooy Kan, M. Eggink, J. H. M. Frijns, K. M. Stankovic, Progression of contralateral hearing loss in patients with sporadic vestibular schwannoma. Front. Neurol. 11, 796 (2020).
22 L. Koelman, O. Pivovarova-Ramich, A. F. H. Pfeiffer, T. Grune, K. Aleksandrova, Cytokines for evaluation of chronic inflammatory status in ageing research: Reliability and phenotypic characterisation. Immun. Ageing 16, 11 (2019).
23 S. E. O'Bryant, S. Lista, R. A. Rissman, M. Edwards, F. Zhang, J. Hall, H. Zetterberg, S. Lovestone, V. Gupta, N. Graff-Radford, R. Martins, A. Jeromin, S. Waring, E. Oh, M. Kling, L. D. Baker, H. Hampel, Comparing biological markers of Alzheimer's disease across blood fraction and platforms: Comparing apples to oranges. Alzheimer's Dement. 3, 27-34 (2016).
24 W. Hong, M. Zhao, H. Li, F. Peng, F. Wang, N. Li, H. Xiang, Y. Su, Y. Huang, S. Zhang, G. Zhao, R. Zhou, L. Mao, Z. Lin, Y. Fang, Q. Zhang, B. Xie, Higher plasma S100B concentrations in schizophrenia patients, and dependently associated with inflammatory markers. Sci. Rep. 6, 27584 (2016).
25 S. Sugie, S. Mukai, K. Yamasaki, T. Kamibeppu, H. Tsukino, T. Kamoto, Plasma macrophage-stimulating protein and hepatocyte growth factor levels are associated with prostate cancer progression. Hum. Cell 29, 22-29 (2016).
26 M. Beranek, P. Kolar, S. Tschoplova, K. Kankova, A. Vasku, Genetic variation and plasma level of the basic fibroblast growth factor in proliferative diabetic retinopathy. Diabetes Res. Clin. Pract. 79, 362-367 (2008).
27 É. S. Vanamee, D. L. Faustman, TNFR2: A novel target for cancer immunotherapy. Trends Mol. Med. 23, 1037-1046 (2017).
28 Y. Wang, P. Li, B. Wang, S. Wang, P. Liu, Identification of myeloid-derived suppressor cells that have an immunosuppressive function in NF2 patients. J. Cancer Res. Clin. Oncol. 145, 523-533 (2019).
29 T. Chinen, A. K. Kannan, A. G. Levine, X. Fan, U. Klein, Y. Zheng, G. Gasteiger, Y. Feng, J. D. Fontenot, A. Y. Rudensky, An essential role for the IL-2 receptor in Tre g cell function. Nat. Immunol. 17, 1322-1333 (2016).
30 P. Chongsathidkiet, C. Jackson, S. Koyama, F. Loebel, X. Cui, S. H. Farber, K. Woroniecka, A. A. Elsamadicy, C. A. Dechant, H. R. Kemeny, L. Sanchez-Perez, T. A. Cheema, N. C. Souders, J. E. Herndon, J.-V. Coumans, J. I. Everitt, B. V. Nahed, J. H. Sampson, M. D. Gunn, R. L. Martuza, G. Dranoff, W. T. Curry, P. E. Fecci, Sequestration of T cells in bone marrow in the setting of glioblastoma and other intracranial tumors. Nat. Med. 24, 1459-1468 (2018).
31 A. Zhang, T. Zou, D. Guo, Q. Wang, Y. Shen, H. Hu, B. Ye, M. Xiang. The immune system can hear noise. Front. Immunol. 11, 619189 (2020).
32 Y. Liu, Y. Cai, L. Liu, Y. Wu, X. Xiong, Crucial biological functions of CCL7 in cancer. Peer J 6, e4928 (2018).
33 A. Zlotnik, O. Yoshie, The chemokine superfamily revisited. Immunity 36, 705-716 (2012).
34 N. B. Sautter, E. H. Shick, R. M. Ransohoff, I. F. Charo, K. Hirose, CC chemokine receptor 2 is protective against noise-induced hair cell death: Studies in CX3CR1+/G F P mice. J. Assoc. Res. Otolaryngol. 7, 361-372 (2006).
35 L. D. Landegger, S. Vasilijic, T. Fujita, V. Y. Soares, R. Seist, L. Xu, K. M. Stankovic, Cytokine levels in inner ear fluid of young and aged mice as molecular biomarkers of noise-induced hearing loss. Front. Neurol. 10, 977 (2019).
36 S. Fujiwara, S. Hoshikawa, T. Ueno, M. Hirata, T. Saito, T. Ikeda, H. Kawaguchi, K. Nakamura, S. Tanaka, T. Ogata, SOX10 transactivates S100B to suppress Schwann cell proliferation and to promote myelination. PLOS ONE 9, e115400 (2014).
37 R. D. S. Doddrell, X.-P. Dun, A. Shivane, M. L. Feltri, L. Wrabetz, M. Wegner, E. Sock, C. O. Hanemann, D. B. Parkinson, Loss of SOX10 function contributes to the phenotype of human Merlin-null schwannoma cells. Brain 136, 549-563 (2013).
38 J. Lin, Q. Yang, Z. Yan, J. Markowitz, P. T. Wilder, F. Carrier, D. J. Weber, Inhibiting S100B restores p53 levels in primary malignant melanoma cancer cells. J. Biol. Chem. 279, 34071-34077 (2004).
39 H. Wang, L. Zhang, I. Y. Zhang, X. Chen, A. Da Fonseca, S. Wu, H. Ren, S. Badie, S. Sadeghi, M. Ouyang, C. D. Warden, B. Badie, S100B promotes glioma growth through chemoattraction of myeloid-derived macrophages. Clin. Cancer Res. 19, 3764-3775 (2013).
40 A. R. Bresnick, D. J. Weber, D. B. Zimmer, S100 proteins in cancer. Nat. Rev. Cancer 15, 96-109 (2015).
41 A. A. Kanner, N. Marchi, V. Fazio, M. R. Mayberg, M. T. Koltz, V. Siomin, G. H. J. Stevens, T. Masaryk, B. Aumayr, M. A. Vogelbaum, G. H. Barnett, D. Janigro, Serum S100B: A noninvasive marker of blood-brain barrier function and brain lesions. Cancer 97, 2806-2813 (2003).
42 C. K. Kandathil, M. E. Cunnane, M. J. McKenna, H. D. Curtin, K. M. Stankovic, Correlation between aspirin intake and reduced growth of human vestibular schwannoma: Volumetric analysis. Otol. Neurotol. 37, 1428-1434 (2016).
43 Committee on Hearing and Equilibrium, Committee on Hearing and Equilibrium guidelines for the evaluation of hearing preservation in acoustic neuroma (vestibular schwannoma). American Academy of Otolaryngology-Head and Neck Surgery Foundation, INC. Otolaryngol. Head Neck Surg. 113, 179-180 (1995).
44 D. W. Hosmer Jr., S. Lemeshow, R. X. Sturdivant, Applied Logistic Regression (John Wiley & Sons, ed. 3, 2013).
45 S. Mazzara, R. L. Rossi, R. Grifantini, S. Donizetti, S. Abrignani, M. Bombaci, CombiROC: An interactive web tool for selecting accurate marker combinations of omics data. Sci. Rep. 7, 45477 (2017).
46 J. T. Aguilan, K. Kulej, S. Sidoli, Guide for protein fold change and p-value calculation for non-experts in proteomics. Mol. Omics 16, 573-582 (2020).

TABLE S1
List and full names of assayed immune factors

| Common | |
|---|---|
| name | Full name |
| APRIL | A proliferation-inducing ligand |
| BAFF | B-cell activating factor |
| BLC | B lymphocyte hemoattractant (CXCL13) |
| CD30 | CD30 |
| CD40L | CD40 ligand |
| ENA-78 | Epithelial neutrophil-activating peptide |
| | 78 (CXCL5) |
| Eotaxin | Eotaxin (CCL11) |
| Eotaxin-2 | Eotaxin-2 (CCL24) |
| Eotaxin-3 | Eotaxin-3 (CCL26) |
| FGF-2 | Fibroblast Growth Factor-2 |
| Fractalkine | Fractalkine (CX3CL1) |
| G-CSF | Granulocyte-colony stimulating factor |
| GM-CSF | Granulocyte macrophage colony-stimulating |
| | factor |
| GRO-a | Growth-related oncogene a (CXCL1) |
| HGF | Hepatocyte Growth Factor |
| IFN-α | Interferon-α |
| IFN-g | Interferon-γ |
| IL-10 | Interleukin-10 |
| IL-12p70 | Interleukin-12p70 |
| IL-13 | Interleukin-13 |
| IL-15 | Interleukin-15 |
| IL-16 | Interleukin-16 |
| IL-17A | Interleukin-17A |
| IL-18 | Interleukin-18 |
| IL-1α | Interleukin-1α |
| IL-1β | Interleukin-1β |
| IL-2 | Interleukin-2 |
| IL-20 | Interleukin-20 |
| IL-21 | Interleukin-21 |
| IL-22 | Interleukin-22 |
| IL-23 | Interleukin-23 |
| IL-27 | Interleukin-27 |
| IL-2R | Interleukin-2R |
| IL-3 | Interleukin-3 |
| IL-31 | Interleukin-31 |
| IL-4 | Interleukin-4 |
| IL-5 | Interleukin-5 |
| IL-6 | Interleukin-6 |
| IL-7 | Interleukin-7 |
| IL-8 | Interleukin-8 |
| IL-9 | Interleukin-9 |
| IP-10 | Interferon-γ inducible protein-10 (CXCL10) |
| I-TAC | Interferon-inducible T Cell Alpha |
| | Chemoattractant (CXCL11) |
| LIF | Leukemia inhibitory factor |
| MCP-1 | Monocyte chemoattractant protein-1 (CCL2) |
| MCP-2 | Monocyte chemoattractant protein-2 (CCL8) |
| MCP-3 | Monocyte chemoattractant protein-3 (CCL7) |
| M-CSF | Macrophage colony-stimulating factor |
| MDC | Macrophage-derived chemokine (CCL22) |
| MIF | Macrophage migration inhibitory factor |
| MIG | Monokine induced by interferon gamma (CXCL9) |
| MIP-1α | Macrophage inflammatory protein-1α (CCL3) |
| MIP-1β | Macrophage inflammatory protein-1β (CCL4) |
| MIP-3α | Macrophage inflammatory protein-3α (CCL20) |
| MMP-1 | Matrix metalloproteinase-1 |
| NGF-β | Nerve Growth Factor-β |
| S100B | S100B |
| SCF | Stem cell factor |
| SDF-1α | Stromal cell-derived factor 1α (CXCL12) |
| TNF-α | Tumor necrosis factor- α |
| TNF- β | Tumor necrosis factor- β |
| TNF-R2 | Tumor necrosis factor- receptor 2 |
| TRAIL | Tumor necrosis factor (TNF)- |
| | related apoptosis-inducing ligand |
| TSLP | Thymic stromal lymphopoietin |
| TWEAK | Tumor necrosis factor-like weak |
| | inducer of apoptosis |
| VEGF-A | Vascular endothelial growth factor-A |

TABLE S2
Detectability of candidate biomarkers in plasma of VS patients

| Candidate | | |
|---|---|---|
| biomarkers | Samples between LLOQ and ULOQ | |
| APRIL | 81/81 | (100%) ^{a} |
| BAFF | 56/81 | (69%) ^{a} |
| BLC | 73/81 | (90%) ^{a} |
| CD30 | 81/81 | (100%) ^{a} |
| CD40L | 32/81 | (40%) ^{a} |
| ENA-78 | 49/81 | (60%) ^{a} |
| Eotaxin | 77/81 | (95%) ^{a} |
| Eotaxin-2 | 80/81 | (99%) ^{a} |
| Eotaxin-3 | 47/81 | (58%) ^{a} |
| FGF-2 | 35/81 | (43%) ^{a} |
| Fractalkine | 10/81 | (12%) ^{a} |
| G-CSF | 6/81 | (7%) ^{a} |
| GM-CSF | 2/81 | (2%) ^{a} |
| GRO-α | 1/81 | (1%) ^{a} |
| HGF | 80/81 | (99%) ^{a} |
| IFN-α | 5/81 | (6%) ^{a} |
| IFN-γ | 21/81 | (26%) ^{a} |
| IL-10 | 16/81 | (20%) ^{a} |
| IL-12p70 | 4/81 | (5%) ^{a} |
| IL-13 | 18/81 | (22%) ^{a} |
| IL-15 | 13/81 | (16%) ^{a} |
| IL-16 | 81/81 | (100%) ^{a} |
| IL-17A | 33/81 | (41%) ^{a} |
| IL-18 | 43/81 | (53%) ^{a} |
| IL-18 | 123/123 | (100%) ^{b} |
| IL-1α | 8/81 | (10%) ^{a} |
| IL-1β | 6/81 | (7%) ^{a} |
| IL-2 | 49/81 | (60%) ^{a} |
| IL-20 | 19/81 | (23%) ^{a} |
| IL-21 | 7/81 | (9%) ^{a} |
| IL-22 | 6/81 | (7%) ^{a} |
| IL-23 | 14/81 | (17%) ^{a} |
| IL-27 | 16/81 | (20%) ^{a} |
| IL-2R | 81/81 | (100%) ^{a} |
| IL-3 | 4/81 | (5%) ^{a} |
| IL-31 | 5/81 | (6%) ^{a} |
| IL-4 | 15/81 | (19%) ^{a} |
| IL-5 | 7/81 | (9%) ^{a} |
| IL-6 | 8/81 | (10%) ^{a} |
| IL-7 | 14/81 | (17%) ^{a} |
| IL-8 | 15/81 | (19%) ^{a} |
| IL-9 | 5/81 | (6%) ^{a} |
| IP-10 | 80/81 | (99%) ^{a} |
| I-TAC | 5/81 | (6%) ^{a} |
| LIF | 18/81 | (22%) ^{a} |
| MCP-1 | 81/81 | (100%) ^{a} |
| MCP-2 | 80/81 | (99%) ^{a} |
| MCP-3 | 79/81 | (98%) ^{a} |
| M-CSF | 8/81 | (10%) ^{a} |
| MDC | 77/81 | (95%) ^{a} |
| MIF | 81/81 | (100%) ^{a} |
| MIG | 30/81 | (37%) ^{a} |
| MIP-1α | 43/81 | (53%) ^{a} |
| MIP-1β | 36/81 | (44%) ^{a} |
| MIP-3α | 5/81 | (6%) ^{a} |
| MMP1 | 7/81 | (9%) ^{a} |
| NGF- β | 10/81 | (12%) ^{a} |
| S100 B | 122/123 | (99%) ^{c} |
| SCF | 19/81 | (23%) ^{a} |
| SDF-1α | 81/81 | (100%) ^{a} |
| TNF-α | 3/81 | (4%) ^{a} |
| TNF-α | 29/123 | (24%) ^{b} |
| TNF- β | 14/81 | (17%) ^{a} |
| TNF-R2 | 81/81 | (100%) ^{a} |
| TRAIL | 49/81 | (60%) ^{a} |
| TSLP | 15/81 | (19%) ^{a} |
| TWEAK | 81/81 | (100%) ^{a} |
| VEGF-A | 76/81 | (94%) ^{a} |

Candidate biomarkers with an absolute concentration between the LLOQ and ULOQ calculated in 75% or more tested plasma samples were included in the study. Twenty of 66 tested candidate biomarkers fulfilled the inclusion criteria (bold). Included candidate biomarkers whose absolute concentration was between 0 pg/mL and LLOQ were assigned the value of the corresponding LLOQ. Candidate biomarkers exceeding ULOQ were approximated with the corresponding ULOQ.
Abbreviations: LLOQ, lower limit of quantification; ULOQ, upper limit of quantification; VS, vestibular schwannoma.
Notes:
^{a} Assessed with Luminex assay;
^{b} Assessed with electrochemiluminescence assay;
^{c} Assessed with ELISA assay.

TABLE S3
The diagnostic power of candidate biomarker panels containing MCP-3 and
S100B assessed in the discovery cohort (CombiROC combinatorial analysis)

| | | | | | Opt |
|---|---|---|---|---|---|
| Biomarker combinations | Symbol | AUC | SE | SP | Cutoff |
| MCP3-S100B-TNFR2-IL2R-BLC-TWEAK | Combo CDXXIV | 1 | 1 | 1 | 0.5 |
| MCP3-S100B-TNFR2-MIF-IL2R-BLC-TWEAK | Combo CDLXXIII | 1 | 1 | 1 | 0.5 |
| MCP3-S100B-TNFR2-MIF-IL2R-BLC-Eotaxin | Combo CDLXXIV | 1 | 1 | 1 | 0.5 |
| MCP3-S100B-TNFR2-CD30-IL2R-BLC-TWEAK | Combo CDLXXIX | 1 | 1 | 1 | 0.5 |
| MCP3-S100B-TNFR2-IL2R-BLC-TWEAK-Eotaxin | Combo CDLXXXIV | 1 | 1 | 1 | 0.5 |
| MCP3-S100B-TNFR2-MIF-CD30-IL2R-BLC-TWEAK | Combo CDXCIV | 1 | 1 | 1 | 0.5 |
| MCP3-S100B-TNFR2-MIF-CD30-IL2R-BLC-Eotaxin | Combo CDXCV | 1 | 1 | 1 | 0.5 |
| MCP3-S100B-TNFR2-MIF-CD30-IL2R-TWEAK-Eotaxin | Combo CDXCVI | 1 | 1 | 1 | 0.5 |
| MCP3-S100B-TNFR2-MIF-IL2R-BLC-TWEAK-Eotaxin | Combo CDXCIX | 1 | 1 | 1 | 0.5 |
| MCP3-S100B-TNFR2-CD30-IL2R-BLC-TWEAK-Eotaxin | Combo D | 1 | 1 | 1 | 0.5 |
| MCP3-S100B-TNFR2-MIF-CD30-IL2R-BLC-TWEAK-Eotaxin | Combo DII | 1 | 1 | 1 | 0.5 |
| MCP3-S100B-TNFR2-MIF-IL2R-Eotaxin | Combo CCCXCVIII | 0.991 | 1 | 0.958 | 0.331 |
| MCP3-S100B-TNFR2-MIF-CD30-IL2R-Eotaxin | Combo CDLXII | 0.991 | 1 | 0.958 | 0.362 |
| MCP3-S100B-TNFR2-MIF-IL2R-TWEAK-Eotaxin | Combo CDLXXV | 0.991 | 1 | 0.958 | 0.284 |
| MCP3-S100B-TNFR2-CD30-IL2R-BLC-Eotaxin | Combo CDLXXX | 0.991 | 0.958 | 0.958 | 0.61 |
| MCP3-S100B-TNFR2-MIF-CD30-BLC-TWEAK-Eotaxin | Combo CDXCVII | 0.986 | 0.958 | 0.958 | 0.456 |
| MCP3-S100B-TNFR2-IL2R-BLC-Eotaxin | Combo CDXXV | 0.984 | 0.958 | 0.958 | 0.636 |
| MCP3-S100B-TNFR2-CD30-IL2R-TWEAK-Eotaxin | Combo CDLXXXI | 0.984 | 0.958 | 0.958 | 0.684 |
| MCP3-S100B-TNFR2-MIF-IL2R-TWEAK | Combo CCCXCVII | 0.983 | 0.958 | 0.917 | 0.295 |
| MCP3-S100B-TNFR2-CD30-IL2R-Eotaxin | Combo CDXIII | 0.983 | 0.958 | 0.958 | 0.612 |
| MCP3-S100B-TNFR2-IL2R-TWEAK-Eotaxin | Combo CDXXVI | 0.981 | 0.958 | 0.958 | 0.619 |
| MCP3-S100B-TNFR2-BLC-TWEAK-Eotaxin | Combo CDXXVII | 0.981 | 0.917 | 0.958 | 0.497 |
| MCP3-S100B-TNFR2-MIF-CD30-IL2R-TWEAK | Combo CDLXI | 0.981 | 0.958 | 0.917 | 0.29 |
| MCP3-S100B-TNFR2-MIF-CD30-TWEAK-Eotaxin | Combo CDLXVI | 0.981 | 1 | 0.833 | 0.186 |
| MCP3-S100B-TNFR2-MIF-BLC-TWEAK-Eotaxin | Combo CDLXXVI | 0.981 | 1 | 0.875 | 0.331 |
| MCP3-S100B-MIF-CD30-IL2R-BLC-TWEAK | Combo CDLXXXVI | 0.981 | 0.958 | 0.917 | 0.473 |
| MCP3-S100B-MIF-IL2R-BLC-TWEAK-Eotaxin | Combo CDXCI | 0.981 | 0.958 | 0.917 | 0.365 |
| MCP3-S100B-MIF-CD30-IL2R-BLC-TWEAK-Eotaxin | Combo DI | 0.981 | 0.958 | 0.875 | 0.382 |
| MCP3-S100B-TNFR2-MIF-CD30-IL2R | Combo CCCLXXIV | 0.979 | 0.917 | 0.958 | 0.663 |
| MCP3-S100B-TNFR2-MIF-CD30-Eotaxin | Combo CCCLXXXII | 0.979 | 1 | 0.833 | 0.207 |
| MCP3-S100B-TNFR2-MIF-CD30-IL2R-BLC | Combo CDLIX | 0.979 | 0.958 | 0.917 | 0.364 |
| MCP3-S100B-TNFR2-MIF-CD30-BLC-TWEAK | Combo CDLXIV | 0.979 | 0.958 | 0.917 | 0.356 |
| MCP3-S100B-TNFR2-CD30-BLC-TWEAK-Eotaxin | Combo CDLXXXII | 0.979 | 0.917 | 0.958 | 0.561 |
| MCP3-S100B-TNFR2-IL2R-Eotaxin | Combo CCCVII | 0.977 | 0.958 | 0.958 | 0.591 |
| MCP3-S100B-TNFR2-MIF-IL2R-BLC | Combo CCCXCV | 0.977 | 0.958 | 0.917 | 0.347 |
| MCP3-S100B-TNFR2-MIF-BLC-Eotaxin | Combo CDI | 0.977 | 1 | 0.833 | 0.172 |
| MCP3-S100B-TNFR2-MIF-TWEAK-Eotaxin | Combo CDII | 0.977 | 0.833 | 1 | 0.843 |
| MCP3-S100B-TNFR2-MIF-Eotaxin | Combo CCLXXI | 0.976 | 0.833 | 1 | 0.866 |
| MCP3-S100B-TNFR2-CD30-Eotaxin | Combo CCXCI | 0.976 | 0.958 | 0.875 | 0.33 |
| MCP3-S100B-TNFR2-MIF-CD30-BLC-Eotaxin | Combo CDLXV | 0.976 | 1 | 0.833 | 0.172 |
| MCP3-S100B-TNFR2-Eotaxin | Combo CLXVI | 0.974 | 0.958 | 0.875 | 0.328 |
| MCP3-S100B-TNFR2-MIF-BLC-TWEAK | Combo CD | 0.974 | 0.917 | 0.958 | 0.493 |
| MCP3-S100B-TNFR2-CD30-BLC-Eotaxin | Combo CDXVI | 0.974 | 0.875 | 0.958 | 0.72 |
| MCP3-S100B-MIF-IL2R-BLC-TWEAK | Combo CDXLV | 0.974 | 0.917 | 0.958 | 0.525 |
| MCP3-S100B-TNFR2-MIF-IL2R | Combo CCLXV | 0.972 | 0.917 | 0.958 | 0.602 |
| MCP3-S100B-TNFR2-BLC-Eotaxin | Combo CCCX | 0.972 | 0.875 | 0.958 | 0.696 |
| MCP3-S100B-TNFR2-TWEAK-Eotaxin | Combo CCCXI | 0.972 | 0.875 | 0.958 | 0.7 |
| MCP3-S100B-TNFR2-CD30-IL2R-BLC | Combo CDX | 0.972 | 0.958 | 0.958 | 0.378 |
| MCP3-S100B-TNFR2-CD30-BLC-TWEAK | Combo CDXV | 0.972 | 0.917 | 0.917 | 0.521 |
| MCP3-S100B-TNFR2-CD30-TWEAK-Eotaxin | Combo CDXVII | 0.972 | 0.875 | 0.958 | 0.699 |
| MCP3-S100B-MIF-BLC-TWEAK-Eotaxin | Combo CDXLVIII | 0.972 | 0.958 | 0.917 | 0.373 |
| MCP3-S100B-MIF-CD30-BLC-TWEAK-Eotaxin | Combo CDLXXXIX | 0.972 | 0.958 | 0.875 | 0.308 |
| MCP3-S100B-TNFR2-IL2R-BLC | Combo CCCIV | 0.97 | 0.958 | 0.958 | 0.397 |
| MCP3-S100B-TNFR2-IL2R-TWEAK | Combo CCCVI | 0.97 | 0.958 | 0.958 | 0.394 |
| MCP3-S100B-TNFR2-MIF | Combo CXXXI | 0.969 | 0.958 | 0.875 | 0.198 |
| MCP3-S100B-TNFR2-BLC | Combo CLXIII | 0.969 | 0.958 | 0.917 | 0.392 |
| MCP3-S100B-TNFR2-MIF-TWEAK | Combo CCLXX | 0.969 | 0.958 | 0.875 | 0.194 |
| MCP3-S100B-TNFR2-BLC-TWEAK | Combo CCCIX | 0.969 | 0.958 | 0.833 | 0.21 |
| MCP3-S100B-TNFR2-MIF-CD30-TWEAK | Combo CCCLXXXI | 0.969 | 0.958 | 0.875 | 0.191 |
| MCP3-S100B-TNFR2-CD30-IL2R-TWEAK | Combo CDXII | 0.969 | 0.958 | 0.958 | 0.404 |
| MCP3-S100B-MIF-CD30-BLC-TWEAK | Combo CDXXXVI | 0.969 | 0.958 | 0.875 | 0.333 |
| MCP3-S100B-TNFR2CD30 | Combo CXLVI | 0.967 | 0.958 | 0.917 | 0.331 |
| MCP3-S100B-TNFR2-TWEAK | Combo CLXV | 0.967 | 0.958 | 0.917 | 0.308 |
| MCP3-S100B-TNFR2-MIF-CD30 | Combo CCLI | 0.967 | 0.958 | 0.875 | 0.199 |
| MCP3-S100B-TNFR2-CD30-TWEAK | Combo CCXC | 0.967 | 0.958 | 0.917 | 0.329 |
| MCP3-S100B-MIF-IL2R-BLC-Eotaxin | Combo CDXLVI | 0.967 | 0.875 | 1 | 0.748 |
| MCP3-S100B-MIF-IL2R-TWEAK-Eotaxin | Combo CDXLVII | 0.967 | 0.917 | 0.958 | 0.523 |
| MCP3-S100B-TNFR2 | Combo LIV | 0.965 | 0.958 | 0.917 | 0.326 |
| MCP3-S100B-TNFR2-MIF-BLC | Combo CCLXVIII | 0.965 | 0.958 | 0.875 | 0.231 |
| MCP3-S100B-TNFR2-CD30IL2R | Combo CCLXXXV | 0.965 | 0.958 | 0.958 | 0.381 |
| MCP3-S100B-TNFR2-CD30-BLC | Combo CCLXXXVIII | 0.965 | 0.958 | 0.917 | 0.392 |
| MCP3-S100B-TNFR2-MIF-CD30-BLC | Combo CCCLXXIX | 0.965 | 0.958 | 0.875 | 0.235 |
| MCP3-S100B-MIF-CD30-IL2R-TWEAK-Eotaxin | Combo CDLXXXVIII | 0.965 | 0.917 | 0.958 | 0.49 |
| MCP3-S100B-MIF-IL2R-TWEAK | Combo CCCXLI | 0.964 | 0.875 | 1 | 0.633 |
| MCP3-S100B-MIF-BLC-TWEAK | Combo CCCXLIV | 0.964 | 0.875 | 0.958 | 0.651 |
| MCP3-S100B-MIF-CD30-IL2R-BLC-Eotaxin | Combo CDLXXXVII | 0.964 | 0.875 | 1 | 0.749 |
| MCP3-S100B-MIF-CD30-IL2R-TWEAK | Combo CDXXXIII | 0.962 | 0.875 | 1 | 0.618 |
| MCP3-S100B-TNFR2-IL2R | Combo CLX | 0.96 | 0.958 | 0.958 | 0.414 |
| MCP3-S100B-MIF-BLC-Eotaxin | Combo CCCXLV | 0.96 | 0.875 | 0.958 | 0.683 |
| MCP3-S100B-MIF-IL2R-Eotaxin | Combo CCCXLII | 0.958 | 0.875 | 1 | 0.745 |
| MCP3-S100B-MIF-CD30-BLC-Eotaxin | Combo CDXXXVII | 0.957 | 0.875 | 0.958 | 0.696 |
| MCP3-S100B-MIF-TWEAK | Combo CC | 0.955 | 0.833 | 1 | 0.722 |
| MCP3-S100B-MIF-Eotaxin | Combo CCI | 0.955 | 0.875 | 0.958 | 0.599 |
| MCP3-S100B-MIF-CD30-TWEAK | Combo CCCXXV | 0.955 | 0.833 | 1 | 0.723 |
| MCP3-S100B-MIF-CD30-IL2R-Eotaxin | Combo CDXXXIV | 0.955 | 0.875 | 1 | 0.777 |
| MCP3-S100B-MIF-IL2R | Combo CXCV | 0.953 | 0.875 | 1 | 0.676 |
| MCP3-S100B-MIF-BLC | Combo CXCVIII | 0.953 | 0.875 | 1 | 0.739 |
| MCP3-S100B-MIF-CD30-BLC | Combo CCCXXIII | 0.953 | 0.875 | 1 | 0.748 |
| MCP3-S100B-MIF-TWEAK-Eotaxin | Combo CCCXLVI | 0.953 | 0.833 | 1 | 0.753 |
| MCP3-S100B-MIF-CD30-IL2R-BLC | Combo CDXXXI | 0.953 | 0.875 | 0.958 | 0.66 |
| MCP3-S100B-MIF-CD30-TWEAK-Eotaxin | Combo CDXXXVIII | 0.953 | 0.833 | 1 | 0.754 |
| MCP3-S100B-MIF-CD30-Eotaxin | Combo CCCXXVI | 0.951 | 0.833 | 1 | 0.768 |
| MCP3-S100B-MIF-CD30IL2R | Combo CCCXX | 0.95 | 0.875 | 1 | 0.639 |
| MCP3-S100B-MIF | Combo LXXV | 0.948 | 0.875 | 0.958 | 0.633 |
| MCP3-S100B-MIFCD30 | Combo CLXXXI | 0.946 | 0.875 | 1 | 0.662 |
| MCP3-S100B-CD30-IL2R-Eotaxin | Combo CCCLVII | 0.938 | 0.917 | 0.833 | 0.431 |
| MCP3-S100B-CD30-BLC-TWEAK-Eotaxin | Combo CDLIV | 0.938 | 0.833 | 0.958 | 0.716 |
| MCP3-S100B-MIF-IL2R-BLC | Combo CCCXXIX | 0.936 | 0.875 | 0.958 | 0.583 |
| MCP3-S100B-CD30-IL2R-TWEAK-Eotaxin | Combo CDLIII | 0.936 | 0.917 | 0.833 | 0.426 |
| MCP3-S100B-CD30-IL2R-BLC-TWEAK-Eotaxin | Combo CDXCII | 0.934 | 0.833 | 0.958 | 0.676 |
| MCP3-S100B-IL2R-BLC-TWEAK-Eotaxin | Combo CDLVI | 0.932 | 0.833 | 0.958 | 0.66 |
| MCP3-S100B-IL2R-BLC-Eotaxin | Combo CCCLXIX | 0.931 | 1 | 0.75 | 0.228 |
| MCP3-S100B-CD30-IL2R-BLC-Eotaxin | Combo CDLII | 0.931 | 0.917 | 0.833 | 0.426 |
| MCP3-S100B-CD30-Eotaxin | Combo CCXXI | 0.929 | 0.875 | 0.917 | 0.505 |
| MCP3-S100B-CD30-BLC-Eotaxin | Combo CCCLX | 0.929 | 0.875 | 0.917 | 0.524 |
| MCP3-S100B-CD30-IL2R-BLC | Combo CCCLIV | 0.927 | 0.917 | 0.833 | 0.388 |
| MCP3-S100B-CD30-TWEAK-Eotaxin | Combo CCCLXI | 0.927 | 0.875 | 0.917 | 0.488 |
| MCP3-S100B-BLC-TWEAK-Eotaxin | Combo CCCLXXI | 0.927 | 0.875 | 0.958 | 0.557 |
| MCP3-S100B-IL2R-Eotaxin | Combo CCXXXVII | 0.925 | 0.875 | 0.875 | 0.461 |
| MCP3-S100B-TWEAK-Eotaxin | Combo CCXLI | 0.924 | 1 | 0.708 | 0.193 |
| MCP3-S100B-CD30-BLC-TWEAK | Combo CCCLIX | 0.924 | 0.875 | 0.875 | 0.485 |
| MCP3-S100B-IL2R-TWEAK-Eotaxin | Combo CCCLXX | 0.924 | 0.875 | 0.875 | 0.458 |
| MCP3-S100B-CD30-BLC | Combo CCXVIII | 0.92 | 0.833 | 0.958 | 0.57 |
| MCP3-S100B-BLC-Eotaxin | Combo CCXL | 0.92 | 0.958 | 0.792 | 0.314 |
| MCP3-S100B-CD30-IL2R-BLC-TWEAK | Combo CDLI | 0.92 | 0.875 | 0.875 | 0.359 |
| MCP3-S100B-Eotaxin | Combo CX | 0.918 | 1 | 0.708 | 0.183 |
| MCP3-S100B-CD30-IL2R | Combo CCXV | 0.918 | 0.833 | 0.917 | 0.529 |
| MCP3-S100B-CD30-IL2R-TWEAK | Combo CCCLVI | 0.918 | 0.833 | 0.917 | 0.528 |
| MCP3-S100B-CD30 | Combo XC | 0.913 | 0.833 | 0.917 | 0.541 |
| MCP3-S100B-CD30-TWEAK | Combo CCXX | 0.913 | 0.833 | 0.917 | 0.525 |
| MCP3-S100B-IL2R-BLC-TWEAK | Combo CCCLXVIII | 0.913 | 0.833 | 0.958 | 0.64 |
| MCP3-S100B-IL2R-BLC | Combo CCXXXIV | 0.91 | 0.833 | 0.917 | 0.578 |
| MCP3-S100B-IL2R-TWEAK | Combo CCXXXVI | 0.91 | 0.833 | 0.917 | 0.554 |
| MCP3-S100B-BLC-TWEAK | Combo CCXXXIX | 0.908 | 0.833 | 0.958 | 0.52 |
| MCP3-S100B-IL2R | Combo CIV | 0.906 | 0.833 | 0.917 | 0.54 |
| MCP3-S100B-TWEAK | Combo CIX | 0.884 | 0.792 | 0.917 | 0.553 |
| MCP3-S100B-BLC | Combo CVII | 0.882 | 0.917 | 0.833 | 0.31 |
| MCP3-S100B | Combo XXVI | 0.88 | 0.917 | 0.833 | 0.282 |

9-biomarker panel (combo DII) is in bold text.
Abbreviations: AUC, area under curve; Opt, optimal; SE, sensitivity; SP, specificity.

TABLE S4
The diagnostic power of candidate biomarker panels containing MCP-3 and
S100B assessed in the validation cohort (CombiROC combinatorial analysis)

| | | | | | Opt |
|---|---|---|---|---|---|
| Biomarker combinations | Symbol | AUC | SE | SP | Cutoff |
| MCP3-S100B-TNFR2-MIF-CD30-IL2R-BLC-Eotaxin | Combo CDXCV | 0.879 | 0.938 | 0.688 | 0.312 |
| MCP3-S100B-TNFR2-MIF-CD30-IL2R-Eotaxin | Combo CDLXII | 0.878 | 0.938 | 0.688 | 0.312 |
| MCP3-S100B-TNFR2-MIF-CD30-IL2R-BLC-TWEAK | Combo CDXCIV | 0.876 | 0.875 | 0.844 | 0.434 |
| MCP3-S100B-MIF-IL2R-BLC-TWEAK-Eotaxin | Combo CDXCI | 0.875 | 0.812 | 0.844 | 0.429 |
| MCP3-S100B-TNFR2-MIF-CD30-IL2R-BLC-TWEAK-Eotaxin | Combo DII | 0.874 | 0.906 | 0.75 | 0.384 |
| MCP3-S100B-CD30-IL2R-BLC-TWEAK-Eotaxin | Combo CDXCII | 0.874 | 0.844 | 0.844 | 0.445 |
| MCP3-S100B-TNFR2-MIF-CD30-IL2R-TWEAK-Eotaxin | Combo CDXCVI | 0.874 | 0.906 | 0.75 | 0.392 |
| MCP3-S100B-TNFR2-CD30-IL2R-BLC-TWEAK-Eotaxin | Combo D | 0.873 | 0.844 | 0.812 | 0.439 |
| MCP3-8100B-IL2R-BLC-TWEAK-Eotaxin | Combo CDLVI | 0.873 | 0.781 | 0.844 | 0.444 |
| MCP3-S100B-TNFR2-MIF-IL2R-BLC-Eotaxin | Combo CDLXXIV | 0.873 | 0.969 | 0.656 | 0.274 |
| MCP3-S100B-TNFR2-MIF-IL2R-TWEAK-Eotaxin | Combo CDLXXV | 0.872 | 0.906 | 0.719 | 0.379 |
| MCP3-S100B-MIF-IL2R-TWEAK-Eotaxin | Combo CDXLVII | 0.872 | 0.812 | 0.812 | 0.433 |
| MCP3-S100B-TNFR2-IL2R-TWEAK-Eotaxin | Combo CDXXVI | 0.872 | 0.781 | 0.844 | 0.447 |
| MCP3-S100B-IL2R-TWEAK-Eotaxir | Combo CCCLXX | 0.872 | 0.781 | 0.844 | 0.446 |
| MCP3-S100B-TNFR2-CD30-IL2R-BLC-TWEAK | Combo CDLXXIX | 0.872 | 0.875 | 0.844 | 0.447 |
| MCP3-S100B-MIF-CD30-IL2R-BLC-TWEAK-Eotaxin | Combo DI | 0.871 | 0.844 | 0.844 | 0.431 |
| MCP3-S100B-MIF-CD30-IL2R-TWEAK-Eotaxin | Combo CDLXXXVIII | 0.87 | 0.844 | 0.812 | 0.425 |
| MCP3-S100B-MIF-IL2R-BLC-Eotaxin | Combo CDXLVI | 0.87 | 0.719 | 0.906 | 0.609 |
| MCP3-S100B-MIF-IL2R-BLC-TWEAK | Combo CDXLV | 0.87 | 0.781 | 0.906 | 0.597 |
| MCP3-S100B-TNFR2-MIF-CD30-BLC-TWEAK | Combo CDLXIV | 0.87 | 0.875 | 0.844 | 0.483 |
| MCP3-S100B-TNFR2-MIF-CD30-IL2R-TWEAK | Combo CDLXI | 0.87 | 0.875 | 0.812 | 0.436 |
| MCP3-S100B-TNFR2-MIF-CD30-IL2R-BLC | Combo CDLIX | 0.87 | 0.906 | 0.812 | 0.42 |
| MCP3-S100B-TNFR2-MIF-IL2R-BLC-TWEAK-Eotaxin | Combo CDXCIX | 0.869 | 0.906 | 0.688 | 0.358 |
| MCP3-S100B-TNFR2-IL2R-BLC-TWEAK-Eotaxin | Combo CDLXXXIV | 0.869 | 0.781 | 0.844 | 0.444 |
| MCP3-S100B-TNFR2-CD30-IL2R-TWEAK-Eotaxin | Combo CDLXXXI | 0.869 | 0.844 | 0.812 | 0.42 |
| MCP3-S100B-CD30-IL2R-TWEAK-Eotaxin | Combo CDLIII | 0.869 | 0.844 | 0.812 | 0.421 |
| MCP3-S100B-MIF-CD30-IL2R-BLC-Eotaxin | Combo CDLXXXVII | 0.869 | 0.781 | 0.844 | 0.485 |
| MCP3-S100B-TNFR2-MIF-IL2R-Eotaxin | Combo CCCXCVII | 0.869 | 0.719 | 0.906 | 0.61 |
| MCP3-S100B-IL2R-Eotaxin | Combo CCXXXVII | 0.869 | 0.719 | 0.906 | 0.613 |
| MCP3-S100B-TNFR2-IL2R-TWEAK | Combo CCCVI | 0.869 | 0.781 | 0.875 | 0.588 |
| MCP3-S100B-CD30-IL2R-BLC-Eotaxin | Combo CDLII | 0.868 | 0.906 | 0.688 | 0.335 |
| MCP3-S100B-MIF-CD30-IL2R-Eotaxin | Combo CDXXXIV | 0.868 | 0.781 | 0.844 | 0.499 |
| MCP3-S100B-MIF-IL2R-Eotaxin | Combo CCCXLII | 0.868 | 0.719 | 0.906 | 0.616 |
| MCP3-S100B-TNFR2-IL2R-BLC-TWEAK | Combo CDXXIV | 0.868 | 0.781 | 0.906 | 0.605 |
| MCP3-S100B-MIF-IL2R-TWEAK | Combo CCCXLI | 0.868 | 0.875 | 0.75 | 0.371 |
| MCP3-S100B-TNFR2-MIF-CD30-BLC | Combo CCCLXXIX | 0.868 | 0.906 | 0.812 | 0.467 |
| MCP3-S100B-TNFR2-CD30-IL2R-BLC-Eotaxin | Combo CDLXXX | 0.867 | 0.906 | 0.719 | 0.359 |
| MCP3-S100B-MIF-CD30-IL2R-BLC-TWEAK | Combo CDLXXXVI | 0.867 | 0.875 | 0.844 | 0.446 |
| MCP3-S100B-TNFR2-CD30-IL2R-TWEAK | Combo CDXII | 0.867 | 0.844 | 0.812 | 0.45 |
| MCP3-S100B-IL2R-TWEAK | Combo CCXXXVI | 0.867 | 0.906 | 0.75 | 0.344 |
| MCP3-S100B-TNFR2-MIF-CD30-TWEAK | Combo CCCLXXXI | 0.867 | 0.844 | 0.844 | 0.503 |
| MCP3-S100B-TNFR2-CD30-IL2R-BLC | Combo CDX | 0.867 | 0.875 | 0.812 | 0.433 |
| MCP3-S100B-TNFR2-CD30IL2R | Combo CCLXXXV | 0.867 | 0.844 | 0.812 | 0.445 |
| MCP3-S100B-TNFR2-CD30-TWEAK-Eotaxin | Combo CDXVII | 0.866 | 0.781 | 0.844 | 0.486 |
| MCP3-S100B-TNFR2-MIF-TWEAK-Eotaxin | Combo CDII | 0.866 | 0.781 | 0.812 | 0.559 |
| MCP3-S100B-TNFR2-IL2R-BLC-Eotaxin | Combo CDXXV | 0.866 | 0.719 | 0.906 | 0.628 |
| MCP3-S100B-CD30-IL2R-Eotaxin | Combo CCCLVII | 0.866 | 0.906 | 0.688 | 0.332 |
| MCP3-S100B-TNFR2-IL2R-Eotaxin | Combo CCCVII | 0.866 | 0.719 | 0.938 | 0.663 |
| MCP3-S100B-CD30-IL2R-BLC-TWEAK | Combo CDLI | 0.866 | 0.906 | 0.781 | 0.364 |
| MCP3-S100B-IL2R-BLC-TWEAK | Combo CCCLXVIII | 0.866 | 0.875 | 0.781 | 0.406 |
| MCP3-S100B-TNFR2-MIF-IL2R-TWEAK | Combo CCCXCVII | 0.866 | 0.781 | 0.875 | 0.549 |
| MCP3-S100B-TNFR2-MIF-CD30 | Combo CCLI | 0.866 | 0.844 | 0.844 | 0.507 |
| MCP3-S100B-IL2R-BLC-Eotaxin | Combo CCCLXIX | 0.865 | 0.719 | 0.906 | 0.621 |
| MCP3-S100B-TNFR2-CD30-IL2R-Eotaxin | Combo CDXII | 0.865 | 0.906 | 0.719 | 0.355 |
| MCP3-S100B-TNFR2-CD30-BLC-Eotaxin | Combo CDXVI | 0.864 | 0.75 | 0.875 | 0.648 |
| MCP3-S100B-TNFR2-CD30-Eotaxin | Combo CCXCI | 0.864 | 0.75 | 0.875 | 0.648 |
| MCP3-S100B-TNFR2-MIF-Eotaxin | Combo CCLXXI | 0.864 | 0.781 | 0.812 | 0.568 |
| MCP3-S100B-TNFR2-MIF-IL2R-BLC-TWEAK | Combo CDLXXIII | 0.864 | 0.812 | 0.875 | 0.531 |
| MCP3-S100B-CD30-BLC-TWEAK | Combo CCCLIX | 0.864 | 0.906 | 0.812 | 0.463 |
| MCP3-S100B-MIF-CD30-BLC-TWEAK-Eotaxin | Combo CDLXXXIX | 0.863 | 0.781 | 0.844 | 0.509 |
| MCP3-S100B-TNFR2-CD30-BLC-TWEAK-Eotaxin | Combo CDLXXXII | 0.863 | 0.781 | 0.844 | 0.506 |
| MCP3-S100B-TNFR2-MIF-BLC-TWEAK-Eotaxin | Combo CDLXXVI | 0.863 | 0.75 | 0.844 | 0.563 |
| MCP3-S100B-TNFR2-MIF-CD30-TWEAK-Eotaxin | Combo CDLXVI | 0.863 | 0.875 | 0.75 | 0.382 |
| MCP3-S100B-MIF-CD30-TWEAK-Eotaxin | Combo CDXXXVIII | 0.863 | 0.781 | 0.844 | 0.512 |
| MCP3-S100B-CD30-TWEAK-Eotaxin | Combo CCCLXI | 0.863 | 0.781 | 0.844 | 0.499 |
| MCP3-S100B-TWEAK-Eotaxin | Combo CCXLI | 0.863 | 0.75 | 0.875 | 0.599 |
| MCP3-S100B-MIF-CD30-BLC-Eotaxin | Combo CDXXXVII | 0.863 | 0.781 | 0.844 | 0.5 |
| MCP3-S100B-TNFR2-MIF-CD30-Eotaxin | Combo CCCLXXXII | 0.863 | 0.875 | 0.75 | 0.388 |
| MCP3-S100B-TNFR2-CD30-BLC-TWEAK | Combo CDXV | 0.863 | 0.844 | 0.875 | 0.518 |
| MCP3-S100B-CD30-BLC-TWEAK-Eotaxin | Combo CDLIV | 0.862 | 0.781 | 0.844 | 0.502 |
| MCP3-S100B-MIF-BLC-TWEAK-Eotaxin | Combo CDXLVIII | 0.862 | 0.75 | 0.906 | 0.637 |
| MCP3-S100B-TNFR2-MIF-CD30-BLC-Eotaxin | Combo CDLXV | 0.862 | 0.875 | 0.75 | 0.39 |
| MCP3-S100B-CD30-BLC-Eotaxin | Combo CCCLX | 0.862 | 0.781 | 0.844 | 0.491 |
| MCP3-S100B-TNFR2-MIF-BLC-Eotaxin | Combo CDI | 0.862 | 0.75 | 0.844 | 0.575 |
| MCP3-S100B-BLC-Eotaxin | Combo CCXL | 0.862 | 0.75 | 0.906 | 0.648 |
| MCP3-S100B-MIF-CD30-Eotaxin | Combo CCCXXVI | 0.862 | 0.812 | 0.812 | 0.477 |
| MCP3-S100B-MIF-CD30-BLC-TWEAK | Combo CDXXXVI | 0.862 | 0.875 | 0.844 | 0.48 |
| MCP3-S100B-MIF-CD30-IL2R-TWEAK | Combo CDXXXIII | 0.862 | 0.844 | 0.844 | 0.487 |
| MCP3-S100B-CD30-IL2R-TWEAK | Combo CCCLVI | 0.862 | 0.844 | 0.844 | 0.487 |
| MCP3-S100B-MIF-CD30-IL2R-BLC | Combo CDXXXI | 0.862 | 0.875 | 0.781 | 0.41 |
| MCP3-S100B-TNFR2-MIF-IL2R-BLC | Combo CCCXCV | 0.862 | 0.781 | 0.844 | 0.569 |
| MCP3-S100B-MIF-CD30IL2R | Combo CCCXX | 0.862 | 0.875 | 0.781 | 0.423 |
| MCP3-S100B-TNFR2-BLC-TWEAK-Eotaxin | Combo CDXXVII | 0.861 | 0.75 | 0.906 | 0.647 |
| MCP3-S100B-CD30-Eotaxin | Combo CCXXI | 0.861 | 0.781 | 0.844 | 0.515 |
| MCP3-S100B-TNFR2-Eotaxin | Combo CLXVI | 0.861 | 0.75 | 0.906 | 0.649 |
| MCP3-S100B-TNFR2-MIF-IL2R | Combo CCLXV | 0.861 | 0.812 | 0.812 | 0.5 |
| MCP3-S100B-BLC-TWEAK-Eotaxin | Combo CCCLXXI | 0.86 | 0.75 | 0.875 | 0.617 |
| MCP3-S100B-MIF-BLC-Eotaxin | Combo CCCXLV | 0.86 | 0.75 | 0.906 | 0.649 |
| MCP3-S100B-TNFR2-IL2R-BLC | Combo CCCIV | 0.86 | 0.781 | 0.875 | 0.618 |
| MCP3-S100B-CD30-IL28 | Combo CCXV | 0.86 | 0.875 | 0.781 | 0.422 |
| MCP3-S100B-TNFR2-MIF-CD30-BLC-TWEAK-Eotaxin | Combo CDXCVII | 0.859 | 0.875 | 0.75 | 0.386 |
| MCP3-S100B-MIF-TWEAK-Eotaxin | Combo CCCXLVI | 0.859 | 0.75 | 0.875 | 0.611 |
| MCP3-S100B-TNFR2-TWEAK-Eotaxin | Combo CCCXI | 0.859 | 0.75 | 0.906 | 0.648 |
| MCP3-S100B-TNFR2-BLC-Eotaxin | Combo CCCX | 0.859 | 0.75 | 0.906 | 0.648 |
| MOP3-S100B-TNFR2-MIF-BLC-TWEAK | Combo CD | 0.859 | 0.844 | 0.812 | 0.527 |
| MCP3-S100B-TNFR2-IL2R | Combo CLX | 0.859 | 0.781 | 0.875 | 0.62 |
| MCP3-S100B-CD30-IL2R-BLC | Combo CCCLIV | 0.858 | 0.875 | 0.781 | 0.409 |
| MCP3-S100B-TNFR2-MIF-CD30-IL2R | Combo CCCLXXIV | 0.858 | 0.875 | 0.875 | 0.472 |
| MCP3-S100B-MIF-Eotaxin | Combo CCI | 0.857 | 0.75 | 0.875 | 0.626 |
| MCP3-S100B-TNFR2-BLC-TWEAK | Combo CCCIX | 0.857 | 0.812 | 0.875 | 0.602 |
| MCP3-S100B-TNFR2-TWEAK | Combo CLXV | 0.857 | 0.812 | 0.844 | 0.577 |
| MCP3-S100B-TNFR2-CD30-TWEAK | Combo CCXC | 0.856 | 0.812 | 0.875 | 0.579 |
| MCP3-S100B-CD30-TWEAK | Combo CCXX | 0.856 | 0.875 | 0.781 | 0.476 |
| MCP3-S100B-Eotaxin | Combo CX | 0.855 | 0.781 | 0.844 | 0.57 |
| MCP3-S100B-TNFR2-MIF-TWEAK | Combo CCLXX | 0.855 | 0.844 | 0.781 | 0.5 |
| MCP3-S100B-MIF-CD30-TWEAK | Combo CCCXXV | 0.854 | 0.812 | 0.844 | 0.534 |
| MCP3-S100B-TNFR2-CD30-BLC | Combo CCLXXXVIII | 0.854 | 0.781 | 0.875 | 0.564 |
| MCP3-S100B-MIF-IL2R | Combo CXCV | 0.854 | 0.812 | 0.781 | 0.451 |
| MCP3-S100B-TNFR2CD30 | Combo CXLVI | 0.854 | 0.812 | 0.844 | 0.567 |
| MCP3-S100B-TNFR2-MIF | Combo CXXXI | 0.854 | 0.875 | 0.781 | 0.503 |
| MCP3-S100B-TNFR2 | Combo LIV | 0.853 | 0.812 | 0.812 | 0.579 |
| MCP3-S100B-IL2R-BLC | Combo CCXXXIV | 0.852 | 0.875 | 0.719 | 0.327 |
| MCP3-S100B-MIF-CD30-BLC | Combo CCCXXIII | 0.852 | 0.875 | 0.812 | 0.491 |
| MCP3-S100B-CD30 | Combo XC | 0.851 | 0.875 | 0.812 | 0.513 |
| MCP3-S100B-CD30-BLC | Combo CCXVIII | 0.849 | 0.875 | 0.812 | 0.508 |
| MCP3-S100B-TNFR2-MIF-BLC | Combo CCLXVIII | 0.849 | 0.812 | 0.812 | 0.563 |
| MCP3-S100B-MIFCD30 | Combo CLXXXI | 0.849 | 0.875 | 0.75 | 0.435 |
| MCP3-S100B-TNFR2-BLC | Combo CLXIII | 0.847 | 0.812 | 0.812 | 0.59 |
| MCP3-S100B-IL2R | Combo CIV | 0.846 | 0.844 | 0.781 | 0.509 |
| MCP3-S100B-MIF-BLC-TWEAK | Combo CCCXLIV | 0.84 | 0.844 | 0.75 | 0.484 |
| MCP3-S100B-MIF-TWEAK | Combo CC | 0.84 | 0.844 | 0.75 | 0.493 |
| MCP3-S100B-MIF-IL2R-BLC | Combo CCCXXIX | 0.84 | 0.875 | 0.719 | 0.407 |
| MCP3-S100B-MIF-BLC | Combo CXCVIII | 0.839 | 0.812 | 0.75 | 0.497 |
| MCP3-S100B-MIF | Combo LXXV | 0.834 | 0.812 | 0.75 | 0.505 |
| MCP3-S100B-BLC-TWEAK | Combo CCXXXIX | 0.829 | 0.875 | 0.75 | 0.478 |
| MCP3-S100B-TWEAK | Combo CIX | 0.827 | 0.906 | 0.688 | 0.399 |
| MCP3-S100B-BLC | Combo CVII | 0.803 | 0.938 | 0.625 | 0.346 |
| MCP3-S100B | Combo XXVI | 0.792 | 0.906 | 0.719 | 0.476 |

9-biomarker panel (combo DII) is in bold text.
Abbreviations: AUC, area under curve; Opt, optimal; SE, sensitivity; SP, specificity.

TABLE S5
Combinatorial analysis of candidate biomarkers
(logistic regression analysis)

| | | | | |
|---|---|---|---|---|
| | | | | The significance |
| | | | | level of AUC |
| | | | | difference |
| | | | | compared to the |
| Biomarker | AUC | SE | 95% CI | 9-panel AUC |

Discovery cohort

| | | | | |
|---|---|---|---|---|
| TNF-R2 | 0.943 | 0.0309 | 0.835 to 0.989 | P = 0.0651 |
| MIF | 0.931 | 0.0396 | 0.819 to 0.984 | P = 0.0814 |
| CD30 | 0.872 | 0.0525 | 0.743 to 0.951 | P = 0.0148 |
| MCP-3 | 0.862 | 0.0602 | 0.732 to 0.944 | P = 0.0219 |
| IL-2R | 0.84 | 0.0595 | 0.706 to 0.930 | P = 0.0072 |
| BLC | 0.836 | 0.0602 | 0.701 to 0.927 | P = 0.0064 |
| TWEAK | 0.786 | 0.0665 | 0.644 to 0.891 | P = 0.0013 |
| Eotaxin | 0.752 | 0.0717 | 0.606 to 0.865 | P = 0.0005 |
| S100B | 0.729 | 0.074 | 0.582 to 0.847 | P = 0.0003 |
| 9-biomarker panel | 1 | 0 | 0.926 to 1.000 | |
| (MCP3/S100B/ | | | | |
| TNFR2/MIF/ | | | | |
| CD30/IL2R/ | | | | |
| BLC/TWEAK/ | | | | |
| Eotaxin) | | | | |

Validation cohort

| | | | | |
|---|---|---|---|---|
| TNF-R2 | 0.814 | 0.0554 | 0.698 to 0.901 | P = 0.2739 |
| MIF | 0.786 | 0.0598 | 0.666 to 0.879 | P = 0.1544 |
| CD30 | 0.782 | 0.0583 | 0.661 to 0.876 | P = 0.1325 |
| MCP-3 | 0.776 | 0.0588 | 0.655 to 0.871 | P = 0.1144 |
| IL-2R | 0.771 | 0.0622 | 0.648 to 0.866 | P = 0.1126 |
| BLC | 0.762 | 0.0598 | 0.639 to 0.859 | P = 0.0796 |
| TWEAK | 0.733 | 0.0628 | 0.608 to 0.836 | P = 0.0376 |
| Eotaxin | 0.717 | 0.0659 | 0.590 to 0.822 | P = 0.0267 |
| S100B | 0.697 | 0.067 | 0.570 to 0.806 | P = 0.0146 |
| 9-biomarker panel | 0.89 | 0.0419 | 0.786 to 0.954 | |
| (MCP3/S100B/ | | | | |
| TNFR2/MIF/ | | | | |
| CD30/IL2R/ | | | | |
| BLC/TWEAK/ | | | | |
| Eotaxin) | | | | |

Significant differences between 9-panel AUC and individual biomarker AUC are in bold text.
Abbreviations: AUC, area under curve; CI, confidence interval; SE, standard error.

What is claimed is:

1. A method of diagnosing and treating vestibular schwannoma in a patient, the method comprising:
obtaining a biological sample from the patient;
measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample,
wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma;
wherein an increased level of eotaxin in the biological sample compared to a reference value range for the level of eotaxin, if the patient, is male, indicates that the patient has vestibular schwannoma;
wherein an increased level of MCP-3 in the biological sample compared to a reference value range for the level of MCP-3 indicates that the patient has serviceable hearing and word recognition, and wherein a decreased level of MCP-3 in the biological sample compared to the reference value range for the level of MCP-3 indicates that the patient has loss of hearing and word recognition;
wherein an increased level of S100B in the biological sample compared to a reference value range for the level of S100B correlates with increased vestibular schwannoma tumor volume; and
treating the patient for the vestibular schwannoma if the patient has a positive diagnosis for vestibular schwannoma.

2. The method of claim 1, wherein the vestibular schwannoma is sporadic vestibular schwannoma.

3. The method of claim 1 or 2, wherein the biological sample is blood or plasma.

4. The method of any one of claims 1-3, wherein the levels of at least 2, at least 3, at least 4, at least 5, at least 6, at least 7, at least 8, or at least 9 of the biomarkers are measured in the biological sample.

5. The method of claim 4, wherein the levels of TNF-R2, MIF, CD30, MCP-3, IL-2R, BLC, TWEAK, eotaxin, and S100B are measured in the biological sample.

6. The method of claim 4, wherein the levels of TNF-R2, MCP-3, and S100B are measured in the biological sample.

7. The method of any one of claims 1-6, further comprising measuring levels of monocyte chemoattractant protein-2 (MCP-2) and stromal cell-derived factor 1a (SDF-1a) if the patient is male, wherein increased levels of MCP-2 and SDF-1a in the biological sample compared to reference value ranges for the levels of MCP-2 and SDF-1a in combination with increased levels of the one or more biomarkers selected from MCP-3, CD30, IL-2R, TWEAK, TNF-R2, S100B, MIF, BLC, and eotaxin in the biological sample compared to the reference value ranges for the levels of the one or more biomarkers selected from MCP-3, CD30, IL-2R, TWEAK, TNF-R2, S100B, MIF, BLC, and eotaxin indicate that the patient has vestibular schwannoma.

8. The method of any one of claims 1-7, further comprising measuring a level of A proliferation-inducing ligand (APRIL) if the patient is female, wherein an increased level of APRIL in the biological sample compared to a reference value range for the level of APRIL in combination with increased levels of the one or more biomarkers selected from MCP-3, CD30, IL-2R, TWEAK, TNF-R2, S100B, MIF, and BLC in the biological sample compared to the reference value ranges for the levels of the one or more biomarkers selected from MCP-3, CD30, IL-2R, TWEAK, TNF-R2, S100B, MIF, and BLC indicate that the patient has vestibular schwannoma.

9. The method of any one of claims 1-8, wherein said treating the patient for the vestibular schwannoma comprises microsurgical resection of the vestibular schwannoma tumor, radiosurgery, stereotactic radiation therapy, or a combination thereof.

10. The method of claim 9, wherein the surgical resection is performed using a ranslabyrinthine, retrosigmoid, or middle cranial fossa incision.

11. The method of any one of claims 1-10, further comprising performing medical imaging of the vestibular schwannoma tumor if the level of S100B indicates that the patient has the vestibular schwannoma tumor with a volume large enough to affect hearing function or damage the cochlear nerve, or if the level of MCP-3 indicates that the patient has loss of hearing or word recognition.

12. The method of claim 11, wherein the microsurgical resection of the vestibular schwannoma tumor is performed if the medical imaging and the level of S100B indicates that the patient has a vestibular schwannoma tumor with a volume large enough to affect hearing function or damage the cochlear nerve.

13. The method of any one of claims 1-12, further comprising performing a hearing test on the patient if the level of MCP-3 indicates that the patient has loss of hearing and word recognition.

14. The method of any one of claims 9-13, wherein the microsurgical resection of the vestibular schwannoma tumor is performed if the level of MCP-3 indicates that the patient has loss of hearing and word recognition.

15. The method of any one of claims 1-14, wherein said measuring comprises performing an electrochemiluminescence-based immunoassay, an enzyme-linked immunosorbent assay (ELISA), a radioimmunoassay (RIA), an immunofluorescent assay (IFA), immunohistochemistry, a Western Blot, an aptamer-based proteomic assay, mass spectrometry, liquid chromatography-tandem mass spectrometry, tandem mass spectrometry, an enzymatic or biochemical assay, liquid chromatography, or nuclear magnetic resonance (NMR).

16. The method of any one of claims 1-15, wherein said measuring comprises performing a multiplex immunoassay.

17. The method of any one of claims 1-16, wherein the subject has not yet developed clinical symptoms.

18. The method of any one of claims 1-16, wherein the subject has developed clinical symptoms.

19. A method of monitoring hearing loss in a patient who has vestibular schwannoma, the method comprising:
obtaining a first biological sample from the patient at a first time point and a second biological sample from the patient later at a second time point;
measuring levels of MCP-3 in the first biological sample and the second biological sample, wherein detection of a decreased level of the MCP-3 in the second biological sample compared to the first biological sample indicate that the patient's hearing and word recognition are worsening, and wherein detection of an increased level of the MCP3 in the biological sample compared to the first biological sample indicate that the patient's hearing and word recognition are improving; and
testing the patient's hearing and word recognition if the level of the MCP3 in the second biological sample indicates that the patient's hearing and word recognition are worsening.

20. The method of claim 19, further comprising performing medical imaging of the patient's vestibular schwannoma tumor if the level of the MCP3 in the second biological sample indicates the patient's hearing and word recognition are worsening.

21. The method of claim 19 or 20, further comprising performing microsurgical resection of the vestibular schwannoma tumor, radiosurgery, stereotactic radiation therapy, or a combination thereof if the patient's hearing and word recognition are worsening.

22. A method of monitoring tumor volume in a patient who has vestibular schwannoma, the method comprising:
obtaining a first biological sample from the patient at a first time point and a second biological sample from the patient later at a second time point;
measuring levels of S100B in the first biological sample and the second biological sample, wherein detection of an increased level of the S100B in the second biological sample compared to the first biological sample indicate that the tumor volume is increasing, and wherein detection of a decreased level of the S100B in the biological sample compared to the first biological sample indicate that the tumor volume is not increasing; and
performing medical imaging of the vestibular schwannoma tumor if the level of the S100B in the second biological sample indicates that the patient's vestibular schwannoma tumor volume is increasing.

23. The method of claim 22, further comprising performing microsurgical resection of the vestibular schwannoma tumor, radiosurgery, stereotactic radiation therapy, or a combination thereof if the level of the S100B and the medical imaging indicate that the patient's tumor volume is increasing.

24. A kit for diagnosing vestibular schwannoma comprising instructions for determining whether a subject has vestibular schwannoma and agents for detecting at least 3 biomarkers selected from the group consisting of tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B).

25. The kit of claim 24, wherein the kit comprises agents for detecting TNF-R2, MIF, CD30, MCP-3, IL-2R, BLC, TWEAK, eotaxin, and S100B.

26. The kit of claim 24, wherein the kit comprises agents for detecting TNF-R2, MCP-3, and S100B.

27. The kit of any one of claims 24-26, further comprising agents for detecting one or more biomarkers selected from monocyte chemoattractant protein-2 (MCP-2), SDF-1a, and APRIL.

28. The kit of any one of claims 24-27, further comprising reagents for performing an immunoassay or aptamer assay.

29. The kit of claim 28, wherein the immunoassay is an electrochemiluminescence-based immunoassay, an enzyme-linked immunosorbent assay (ELISA), a radioimmunoassay (RIA), or an immunofluorescent assay (IFA).

30. The kit of claim 28 or 29, wherein the kit comprises an aptamer or antibody that specifically binds to TNF-R2, an aptamer or antibody that specifically binds to MCP-3, and an aptamer or antibody that specifically binds to S100B.

31. The kit of claim 30, wherein the kit further comprises an aptamer or antibody that specifically binds to MIF, an aptamer or antibody that specifically binds to CD30, an aptamer or antibody that specifically binds to IL-2R, an aptamer or antibody that specifically binds to BLC, an aptamer or antibody that specifically binds to TWEAK, and an aptamer or antibody that specifically binds to eotaxin.

32. A protein selected from the group consisting of tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) for use as a biomarker in diagnosing vestibular schwannoma.

33. An in vitro method of diagnosing vestibular schwannoma, the method comprising:
obtaining a biological sample from the patient;
measuring levels of one or more biomarkers selected from tumor necrosis factor-receptor 2 (TNF-R2), macrophage migration inhibitory factor (MIF), CD30, monocyte chemoattractant protein-3 (MCP-3), interleukin-2R (IL-2R), B lymphocyte chemoattractant (BLC), tumor necrosis factor-like weak inducer of apoptosis (TWEAK), eotaxin, and S100 calcium binding protein B (S100B) in the biological sample,
wherein increased levels of the one or more biomarkers selected from MCP-3, CD30, IL-2R, TWEAK, TNF-R2, S100B, MIF, and BLC in the biological sample compared to reference value ranges for the levels of MCP-3, CD30, IL-2R, TWEAK, TNF-R2, S100B, MIF, and BLC indicate that the patient has vestibular schwannoma;
wherein an increased level of eotaxin in the biological sample compared to a reference value range for the level of eotaxin, if the patient is male, indicates that the patient has vestibular schwannoma;
wherein an increased level of MCP-3 in the biological sample compared to a reference value range for the level of MCP-3 indicates that the patient has serviceable hearing and word recognition, and wherein a decreased level of MCP-3 in the biological sample compared to the reference value range for the level of MCP-3 indicates loss of hearing and word recognition in the patient; and
wherein an increased level of S100B in the biological sample compared to a reference value range for the level of S100B correlates with increased vestibular schwannoma tumor volume.
